(19) United States
(12) Patent Application Publication
(10) Pub. No.: US 2024/0209455 A1
(43) Pub. Date: Jun. 27, 2024

(54) ANALYSIS OF FRAGMENT ENDS IN DNA

(71) Applicants: THE TRANSLATIONAL GENOMICS RESEARCH INSTITUTE, Phoenix, AZ (US); WISCONSIN ALUMNI RESEARCH FOUNDATION, Madison, WI (US)

(72) Inventors: Muhammed Murtaza, Phoenix, AZ (US); Karan K. Budhraja, Madison, WI (US)

(21) Appl. No.: 18/556,737

(22) PCT Filed: Apr. 22, 2022
(86) PCT No.: PCT/US2022/026066

§ 371 Date: Oct. 23, 2023

Related U.S. Application Data
(60) Provisional application No. 63/179,167, filed on Apr. 23, 2021.

(51) Int. Cl. C12Q 1/6886; G16B 20/20; G16B 40/20; G16H 50/20

(52) U.S. Cl. CPC C12Q 1/6886; G16B 20/20; G16B 40/20; G16H 50/20

(57) ABSTRACT

Fragmentation patterns observed in plasma DNA reflect chromatin accessibility in contributing cells. Since DNA shed from cancer cells and blood cells may differ in fragmentation patterns, we investigated whether analysis of genomic positioning and nucleotide sequence at fragment ends can reveal the presence of tumor DNA in blood and aid cancer diagnostics. Whole genome sequencing data from >2700 plasma DNA samples including healthy individuals and patients with 11 different cancer types were analyzed. Higher fractions of fragments with aberrantly positioned ends were observed in patients with cancer, driven by contribution of tumor DNA into plasma. Genome wide analysis of fragment ends using machine learning showed overall area under the receiver operative characteristic curve of 0.96 for detection of cancer. These findings remained robust with as few as 1 million fragments analyzed per sample, indicating that analysis of fragment ends is a cost-effective and accessible approach for cancer detection and monitoring.

### CROSS REFERENCE TO RELATED APPLICATIONS

This application claims the benefit of U.S. Provisional Patent Application No. 63/179,167, filed on Apr. 23, 2021, the contents of which are incorporated herein by reference in its entirety.

### TECHNICAL FIELD

The present invention relates to methods for detecting and quantifying cell-free DNA (cfDNA) in a biological sample to identify a patient's disease and to monitor response to treatment in a patient.

### BACKGROUND

Detection and/or quantitation of certain biomarkers such as cell free DNA (cfDNA) in biological samples like blood, saliva, sputum, stool, urine, cerebral spinal fluid, or tissue can help to diagnose disease, establish a prognosis, and/or aid in selecting or monitoring treatment. For example, the concentration of certain genetic markers in cfDNA can indicate cancer progression or treatment success and can have utility in noninvasive prenatal testing (NIPT) for the detection of trisomy or monosomy, as well as short insertion and deletion mutations in an unborn child (J. Clin. Med. 2014, 3, 537-565). Specific changes in the sequence or modifications of certain DNAs present in stool samples can indicate the presence of colon cancer (Imperiale et al (2014) Multitarget Stool DNA Testing for Colorectal-Cancer Screening. N Engl J Med 2014; 370: 1287-1297).

cfDNA in plasma or serum can be applied as a more specific tumor marker, than conventional biological samples, for the diagnosis and prognosis, as well as the early detection, of cancer. For instance, one study indicates that the elevation of serum cell-free DNA was usually detected in specimens containing elevated tumor markers and is most likely associated with tumor metastases. The electrophoretic pattern of cell-free DNA showed that cell-free DNA from cancer patients is fragmented, containing smaller DNA (100 bp) not found in normal cell-free DNA. Wu, et al. Cell-free DNA: measurement in various carcinomas and establishment of normal reference range. Clin Chim Acta. 2002, 321(1-2):77-87.

Much of the morbidity and mortality of human cancers world-wide is a result of the late diagnosis of these diseases, where treatments are less effective (Torre et al., 2015 CA Cancer J Clin 65:87; and World Health Organization, 2017 Guide to Cancer Early Diagnosis). Unfortunately, clinically proven biomarkers that can be used to broadly diagnose and treat patients are not widely available (Mazzucchelli, 2000 Advances in clinical pathology 4:111; Ruibal Morell, 1992 The International journal of biological markers 7:160; Galli et al., 2013 Clinical chemistry and laboratory medicine 51:1369; Sikaris, 2011 2 Heart, lung &circulation 20:634; Lin et al., 2016 in Screening for Colorectal Cancer: A Systematic Review for the U.S. Preventive Services Task Force. (Rockville, Md.); Wanebo et al., 1978 N Engl J Med 299A48; and Zauber, 2015 Dig Dis Sci 60:681). There is a need for more effective tools and methods to analyze the genetic information available in cfDNA to provide earlier diagnosis and treatment in cancer patients.

### SUMMARY

Analyzing the positioning and nucleotide sequence at fragment ends in plasma DNA may enable cancer diagnostics. In certain aspects, the present invention relates to a method of detecting disease in a patient, the method comprising the steps of: obtaining a sample from the patient: extracting cell-free DNA (cfDNA) from the sample to obtain cfDNA fragments; performing sequencing on the cfDNA fragments extracted from the sample to generate sequencing reads for the cfDNA fragments; determining an average nucleotide frequency at start sites and end sites of the cfDNA fragments; determining a fraction of aberrant fragments in the cfDNA fragments from the sample; inputting the average nucleotide frequency and the fraction of aberrant fragments into a machine learning classifier trained using genomic data from both healthy and diseased subjects; and determining presence of the disease in the patient based on output of the machine learning classifier.

In some aspects, the method further comprises generating the machine learning classifier by training the machine learning classifier using fractions of aberrant fragments in cfDNA from healthy subjects and using fractions of aberrant fragments in cfDNA from diseased subjects.

In other aspects, the method further comprises training the machine learning classifier using average nucleotide frequency at start sites and end sites in cfDNA from healthy subjects and using average nucleotide frequency at start sites and end sites in cfDNA from diseased subjects. In one aspect, the machine learning classifier is trained using genomic data from the earliest available samples from healthy and diseased subjects. In another aspect, the machine learning classifier is trained using genomic data comprising a reference dataset from healthy subjects across age, gender and co-morbidities corresponding with those of the diseased subjects. In yet another aspect, the machine learning classifier is trained using genomic data comprising a dataset from diseased subjects across disease stages and/or disease types.

In some aspects, analysis of as few as one million fragments per sample, as few as 900,000 fragments per sample, as few as 800,000 fragments per sample, as few as 700,000 fragments per sample, as few as 600,000 fragments per sample, or as few as 500,000 fragments per sample from whole genome sequencing libraries allows for detection of the disease.

In other aspects, the disease is cancer. In one aspect, the cancer is a cancer with no established methods for screening selected from the group consisting of cholangiocarcinoma, pancreatic cancer, gastric cancer, and ovarian cancer. In another aspect, the cancer is selected from the group consisting of melanoma, cholangiocarcinoma, glioblastoma, breast cancer, prostate cancer, colorectal cancer, gastric cancer, lung cancer, and ovarian cancer.

In certain aspects, the sample is plasma, urine, or cerebrospinal fluid. In one aspect, the patient is human. In another aspect, the patient is a dog or a cat. In some aspects, the healthy and diseased subjects are non-human. In other aspects, the healthy and diseased subjects include dogs or cats.

In other aspects, the machine learning classifier comprises a random forest, a support vector machine (SVM), a boosting algorithm, a gradient boost method (GBM), an extreme gradient boost method (XGBoost)), and/or a neural network. In one aspect, the machine learning classifier comprises a random forest. In another aspect, the machine learning classifier comprises a gradient boosted tree and/or a neural network. In some aspects, the method is computer-implemented.

In yet other aspects, the present invention relates to a method of detecting disease in a patient, the method comprising the steps of: obtaining a sample from the patient: extracting cell-free DNA (cfDNA) from the sample to obtain cfDNA fragments; performing sequencing on the cfDNA fragments extracted from the sample to generate sequencing reads for the cfDNA fragments; determining a nucleotide frequency at start sites and end sites of the cfDNA fragments; generating a nucleotide frequency vector from the nucleotide frequency at start sites and end sites; determining a fraction of aberrant fragments in the cfDNA fragments from the sample; inputting the nucleotide frequency vector and the fraction of aberrant fragments into a random forest classifier trained using genomic data from both healthy and diseased subjects; and determining presence of the disease in the patient based on output of the random forest classifier.

In one aspect, the method further comprises generating the random forest classifier by training the random forest classifier using fractions of aberrant fragments in cfDNA from healthy subjects and using fractions of aberrant fragments in cfDNA from diseased subjects.

In another aspect, the method further comprises training the random forest classifier using a vector of nucleotide frequency at start sites and end sites in cfDNA from healthy subjects and using a vector of nucleotide frequency at start sites and end sites in cfDNA from diseased subjects.

In yet another aspect, the method further comprises training the random forest classifier using a nucleotide frequency at start sites and end sites in cfDNA from a sample taken from the subject at an earlier point in time. In one aspect, the method further comprises training the random forest classifier using a fraction of aberrant fragments in cfDNA from the sample taken from the subject at the earlier point in time.

In some aspects, the machine learning classifier comprises a random forest, a support vector machine (SVM), a boosting algorithm, a gradient boost method (GBM), an extreme gradient boost method (XGBoost)), and/or a neural network.

In another aspect, the present invention relates to a method of detecting disease in a patient, the method comprising the steps of: obtaining a sample from the patient: extracting cell-free DNA (cfDNA) from the sample to obtain cfDNA fragments; performing sequencing on the cfDNA fragments extracted from the sample to generate sequencing reads for the cfDNA fragments; determining an average nucleotide frequency at start sites and end sites of the cfDNA fragments; determining a fraction of aberrant fragments in the cfDNA fragments from the sample; determining a fraction of short fragments in the cfDNA fragments from the sample; inputting the average nucleotide frequency, the fraction of aberrant fragments, and the fraction of short fragments into a machine learning classifier trained using genomic data from both healthy and diseased subjects; and determining presence of the disease in the patient based on output of the machine learning classifier.

In some aspects, the cfDNA fragments having a length of less than 300 bp, less than 275 bp, less than 250 bp, less than 225 bp, less than 200 bp, less than 175 bp, less than 150 bp, less than 125 bp, or less than 100 bp are considered short fragments. In other aspects, the cfDNA fragments having a length of less than a selected threshold length are considered short fragments. In one aspect, the selected threshold length is about 150 bp.

In other aspects, the present invention relates to a method of detecting disease in a patient, the method comprising the steps of: obtaining a sample from the patient: extracting cell-free DNA (cfDNA) from the sample to obtain cfDNA fragments; performing sequencing on the cfDNA fragments extracted from the sample to generate sequencing reads for the cfDNA fragments; determining an average nucleotide frequency at start sites and end sites of the cfDNA fragments; inputting the average nucleotide frequency into a machine learning classifier trained using genomic data from both healthy and diseased subjects; and determining presence of the disease in the patient based on output of the machine learning classifier.

In some aspects, the method further comprises training the machine learning classifier using average nucleotide frequency at start sites and end sites in cfDNA from healthy subjects and using average nucleotide frequency at start sites and end sites in cfDNA from diseased subjects.

In yet another aspect, the present invention relates to a method of detecting disease in a patient, the method comprising the steps of: obtaining a sample from the patient: extracting cell-free DNA (cfDNA) from the sample to obtain cfDNA fragments; performing sequencing on the cfDNA fragments extracted from the sample to generate sequencing reads for the cfDNA fragments; determining a fraction of aberrant fragments in the cfDNA fragments from the sample; inputting the fraction of aberrant fragments into a machine learning classifier trained using genomic data from both healthy and diseased subjects; and determining presence of the disease in the patient based on output of the machine learning classifier.

In one aspect, the method further comprises generating the machine learning classifier by training the machine learning classifier using fractions of aberrant fragments in cfDNA from healthy subjects and using fractions of aberrant fragments in cfDNA from diseased subjects.

In certain aspects, the disclosed methods further comprises selecting specific nucleotide frequencies to feed into the machine learning classifier by determining which nucleotide frequencies are most highly correlated with tumor fraction and fraction of aberrant fragments (FAF).

In some aspects, the output of the machine learning classifier comprises a probability that the patient has the disease.

In other aspects, the sequencing of the cfDNA fragments is performed with whole genome sequencing and/or hybrid capture sequencing.

Hybrid capture is a form of library enrichment in which a library is probed for known sequences of interest using tagged nucleic acid probes followed by a subsequent “pull-down” of the tagged hybrids: for example, DNA probes tagged with biotin can be efficiently enriched when hybridization is followed by a streptavidin enrichment step. With a “hybrid capture” target enrichment approach, input genomic cfDNA containing aberrant fragments may be enriched (or “captured”) relative to other segments of the genome. Several methodological approaches to target enrichment exist, with multiple commercially available and rigorously optimized kits capable of enriching nearly any well-defined gene target(s).

In yet other aspects, the present invention relates to a non-transitory computer-readable storage device storing computer executable instructions that when executed by a computer control the computer to perform a method for detecting disease in a patient, the method comprising: determining an average nucleotide frequency at start sites and end sites of cfDNA fragments extracted from a sample from the patient; determining a fraction of aberrant fragments in the cfDNA fragments from the sample; inputting the average nucleotide frequency and the fraction of aberrant fragments into a machine learning classifier trained using genomic data from both healthy and diseased subjects; and determining presence of the disease in the patient based on output of the machine learning classifier.

In one aspect, the present invention relates to a non-transitory computer-readable storage device storing computer executable instructions that when executed by a computer control the computer to perform a method for detecting disease in a patient, the method comprising: determining a nucleotide frequency at start sites and end sites of cfDNA fragments extracted from a sample from the patient; generating a nucleotide frequency vector from the nucleotide frequency at start sites and end sites; determining a fraction of aberrant fragments in the cfDNA fragments from the sample; inputting the nucleotide frequency vector and the fraction of aberrant fragments into a random forest classifier trained using genomic data from both healthy and diseased subjects; and determining presence of the disease in the patient based on output of the random forest classifier.

In another aspect, the present invention relates to a non-transitory computer-readable storage device storing computer executable instructions that when executed by a computer control the computer to perform a method for detecting disease in a patient, the method comprising: determining an average nucleotide frequency at start sites and end sites of cfDNA fragments extracted from a sample from the patient; determining a fraction of aberrant fragments in the cfDNA fragments from the sample; determining a fraction of short fragments in the cfDNA fragments from the sample; inputting the average nucleotide frequency, the fraction of aberrant fragments, and the fraction of short fragments into a machine learning classifier trained using genomic data from both healthy and diseased subjects; and determining presence of the disease in the patient based on output of the machine learning classifier.

In yet another aspect, the present invention relates to a non-transitory computer-readable storage device storing computer executable instructions that when executed by a computer control the computer to perform a method for detecting disease in a patient, the method comprising: determining an average nucleotide frequency at start sites and end sites of cfDNA fragments extracted from a sample from the patient; inputting the average nucleotide frequency into a machine learning classifier trained using genomic data from both healthy and diseased subjects; and determining presence of the disease in the patient based on output of the machine learning classifier.

In certain aspects, the present invention relates to a non-transitory computer-readable storage device storing computer executable instructions that when executed by a computer control the computer to perform a method for detecting disease in a patient, the method comprising: determining a fraction of aberrant fragments in cfDNA fragments extracted from a sample from the patient; inputting the fraction of aberrant fragments into a machine learning classifier trained using genomic data from both healthy and diseased subjects; and determining presence of the disease in the patient based on output of the machine learning classifier.

In other aspects, the present invention relates to a computer-implemented system comprising: a server comprising at least one processor configured to generate a machine learning classifier that classifies cfDNA fragment data into a disease classification for a disease, wherein the machine learning classifier is generated by: determining an average nucleotide frequency at start sites and end sites of cfDNA fragments; determining a fraction of aberrant fragments in the cfDNA fragments; and inputting average nucleotide frequencies and fractions of aberrant fragments into the machine learning classifier to train the classifier using genomic data from both healthy and diseased subjects.

### BRIEF DESCRIPTION OF THE DRAWINGS

FIGS. 1A-1F illustrate a fraction of aberrant fragments in plasma samples from patients with cancer. The fraction of aberrant fragments (FAF) was higher in plasma samples from patients with cancer compared to healthy volunteers, in whole genome sequence data from >2700 plasma samples (FIG. 1A). FAF was correlated with tumor fraction measured using copy number analysis in plasma samples. Results from patients with metastatic melanoma are shown in FIG. 1B, and additional results are shown from patients with cholangiocarcinoma (FIG. 3), breast cancer and prostate cancer (FIGS. 7A-7B). Longitudinal changes in FAF during therapy were consistent with changes in tumor fraction measured by copy number analysis in patients with metastatic melanoma. Results from a representative patient are shown in FIG. 1C. Upper panel shows changes in FAF over time and lower panel shows changes in tumor fraction measured using copy number analysis by ichorCNA. Results from additional patients are shown in FIGS. 4A-4C. Despite very low tumor fractions observed in patients with glioblastoma, longitudinal changes in FAF during therapy were consistent with changes in tumor fraction measured using targeted digital sequencing. Results from a representative patient are shown in FIG. 1D, and results from additional patients are shown in FIG. 5. FAF was higher at genomic loci affected by copy number gain in the corresponding tumor genome, compared to unaffected loci or those affected by copy number loss. Results from a representative patient with metastatic melanoma are shown in FIG. 1E, and results from additional patients are shown in FIGS. 6A-6D. For two plasma samples with higher tumor fraction in plasma, we compared FAF between mutated and non-mutated fragments and these results are shown in FIG. 1F.

FIGS. 2A-2D illustrate diagnostic performance for cancer detection using analysis of fragment ends. Results from a random forests classifier trained to distinguish cancer patients from healthy individuals, using fraction of aberrant fragments and average nucleotide frequencies at fragment starts and ends in plasma whole genome sequencing data. For samples in our cohort, overall performance is shown in FIG. 2A, and performance by tumor type is shown in FIG. 2B. For samples in Cristiano et al. (12), overall performance is shown in FIG. 2C, and performance by disease stage is shown in FIG. 2D.

FIG. 3 illustrates a comparison of tumor fraction and FAF in plasma samples from patients with cholangiocarcinoma. Tumor fraction and FAF were correlated with Pearson's r of 0.71 (P=2.2×10−8). On the x-axis, plasma samples with tumor fraction below the limit of detection using ichorCNA are indicated as zero.

FIG. 4 illustrates a comparison of longitudinal changes in tumor fraction and FAF in serial plasma samples from patients with metastatic melanoma, treated on a targeted therapy trial (19). 17 patients from whom at least 4 plasma samples were analyzed and at least one of them had circulating tumor DNA detectable by ichorCNA are included in this figure. For each patient, the top panel shows longitudinal changes in FAF and the bottom panel shows tumor fraction measured using ichorCNA. Days of follow-up are reported since the earliest available blood sample. Shaded areas indicate systemic therapy during the trial. When available, imaging results measured using RECIST are indicated with vertical lines for Stable Disease and with vertical lines for Progressive Disease.

FIG. 5 illustrates a comparison of longitudinal changes in tumor fraction and FAF in serial plasma samples from patients with glioblastoma, treated on a genomics-enabled therapy trial(20). 3 patients from whom at least 4 plasma samples were analyzed are included in this figure. For each patient, the top panel shows longitudinal changes in FAF and the bottom panel shows tumor fraction measured using TARDIS, an assay of patient-specific mutations guided by the patient's own tumor biopsy(34). Days of follow-up are reported since the earliest available blood sample, which was collected prior to surgical resection of the tumor. Subsequent samples were collected after surgical resection and during therapy. Vertical red line indicates clinical disease progression.

FIG. 6 illustrates a comparison of FAF between copy number gain, neutral and loss regions in patients with metastatic melanoma. Density plots for normalized FAF are presented for copy number loss (blue), neutral (purple) and gain regions (red) for 27 plasma samples with at least 20% tumor fraction measured using ichorCNA. Under each plot, p values for comparison of these distributions are presented. GvL: gain regions vs. loss regions. GvN: gain regions vs. neutral regions. LvN: loss regions vs. neutral regions. All 27 samples showed significantly higher FAF in gain regions compared to neutral regions, in gain regions compared to loss regions, or both (P<0.05).

FIGS. 7A and 7B illustrate a comparison of tumor fraction and FAF in plasma samples from patients with metastatic breast and prostate cancer, respectively. Whole genome sequencing data from Adalsteinsson et al. was analyzed for this figure(25). Tumor fraction and FAF were correlated with Pearson's r of 0.66 (P=1.9×10-119) in plasma samples from patients with metastatic breast cancer (A) and with Pearson's r of 0.74 (p=6.8×10-98) in plasma samples from patients with metastatic prostate cancer (B). On the x-axis, plasma samples with tumor fraction below the limit of detection using ichorCNA are indicated as zero.

FIG. 8 illustrates ROC curves for cancer detection by cancer type. Whole genome sequencing data from Cristiano et al. was used to evaluate performance of analysis of fragment ends (27). Each panel shows classifier performance in a cancer subtype. Numbers with brackets are areas under the ROC curves.

FIG. 9 illustrates a co-efficient of variation (CV) for FAF in down-sampled data sets. To calculate CV, multiple independent datasets with decreasing number of DNA fragments were generated and FAF was calculated from these replicates. CVs remained less 1% even for as low as 1 million reads per sample.

FIG. 10 illustrates a classifier performance with down-sampling in our multi-cancer cohort. Down-sampling was performed to limit maximum number of analyzed fragments, as indicated on each panel. Overall classifier performance for cancer detection is shown. Numbers in brackets are area under the ROC curve. Vertical dashed black line indicates 95% specificity.

FIG. 11 illustrates a classifier performance with down-sampling in Cristiano et al.'s published cohort(27). Down-sampling was performed to limit maximum number of analyzed fragments, as indicated on each panel. Overall classifier performance for cancer detection is shown. Numbers in brackets are area under the ROC curve. Vertical dashed black line indicates 95% specificity.

FIG. 12 illustrates an analysis in which for each of 168 features, the correlation between FAF and individual nucleotide frequency was investigated. The x-axis shows the relative position from nucleotide end, where position 11 is the first base of a fragment and position 32 is the last base of a fragment. Some positions showed higher correlation with FAF than others.

FIG. 13 illustrates an analysis in which all 4 nucleotide frequencies from the highest correlation 16 positions (8 from either side) of the cfDNA fragment were fit with a linear regression for FAF using these features, essentially to calculate multivariate correlation coefficients. Certain positions survived multivariate adjustment.

FIG. 14 illustrates multivariate adjusted correlation coefficients sorted in descending order. The top 9 features were chosen to include in a random forest model alongside FAF for cancer detection. These 9 represent 3 loci, −1 position on the fragment start (first base outside the fragment) and +1 and +2 positions on the fragment end (first two bases inside the fragment).

FIG. 15 illustrates a ROC curve for classifier performance using FAF and 9 selected nucleotide frequency features overall.

FIG. 16 illustrates a ROC curve for classifier performance using FAF and 9 selected nucleotide frequency features by stage of cancer.

### DETAILED DESCRIPTION

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

The term “subject” or “patient” as used herein refers to an organism, including, without limitation, humans and other non-human primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some implementations, the subject may be a mammal, preferably a human.

The term “biological sample” refers to a body sample from any animal, but preferably is from a mammal, more preferably from a human. Such samples include biological fluids such as serum, plasma, vitreous fluid, lymph fluid, synovial fluid, follicular fluid, seminal fluid, amniotic fluid, milk, whole blood, urine, cerebrospinal fluid, saliva, sputum, tears, perspiration, mucus, and tissue culture medium, as well as tissue extracts such as homogenized tissue, and cellular extracts. In certain embodiments, blood, serum, plasma, urine and bronchial lavage or other liquid samples are convenient test samples for use in the context of the present methods.

The terms “diagnose” and “detect” are utilized throughout the application in to suggest that a data model that is generated and method determining a probability of the presence of a given physical or medical condition, including but not limited to a cancer, based on a data set related to an individual, referred to herein as a patient. However, the so-called diagnosis provided by aspects of embodiments of the present invention is not analogous to a medical diagnosis, provided by a health professional, often based on the result of a medical text or procedure. Rather, a diagnosis herein is merely a recognition of a pattern, or a given portion of a pattern, where the pattern was generated from a self-learning model, in embodiments of the present invention.

The terms “nucleic acid,” “nucleotide,” “polynucleotide,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The “frequency” of a nucleotide or “nucleotide frequency” refers to a percentage of the number of times a given nucleotide is found at a given position relative to the ends of all analyzed fragments in a sample out of the total number of nucleotides at the same relative position.

The term “fraction of aberrant fragments” refers to the fraction of cfDNA fragments that contain unexpected end sequences. The repositioning of nucleosomes in cancer cells will produce cfDNA fragments that exhibit a higher abundance of fragment start and end sites in unexpected genomic regions. These unexpected genomic regions may include regions that are normally protected by nucleosomes in healthy control samples. Thus, aberrant fragments have start and/or end sites in genomic regions that are not generally observed in healthy control samples.

As used herein, the term “AUC” refers to the Area Under the Curve, for example, of a ROC Curve. That value can assess the merit of a test on a given sample population with a value of 1 representing a good test ranging down to 0.5 which means the test is providing a random response in classifying test subjects. Since the range of the AUC is only 0.5 to 1.0, a small change in AUC has greater significance than a similar change in a metric that ranges for 0 to 1 or 0 to 100%. When the % change in the AUC is given, it will be calculated based on the fact that the full range of the metric is 0.5 to 1.0. A variety of statistics packages can calculate AUC for an ROC curve, such as, JMP™ or Analyse-It™. AUC can be used to compare the accuracy of the classification algorithm across the complete data range. Classification algorithms with greater AUC have, by definition, a greater capacity to classify unknowns correctly between the two groups of interest (disease and no disease). The classification algorithm may be the measure of a single molecule or as complex as the measure and integration of multiple molecules.

As used herein the term, “Receiver Operating Characteristic Curve” or “ROC curve.” is a plot of the performance of a particular feature for distinguishing two populations, patients with cancer, and controls, e.g., those without cancer. Data across the entire population (namely, the patients and controls) are sorted in ascending order based on the value of a single feature. Then, for each value for that feature, the true positive and false positive rates for the data are determined. The true positive rate is determined by counting the number of cases above the value for that feature under consideration and then dividing by the total number of patients. The false positive rate is determined by counting the number of controls above the value for that feature under consideration and then dividing by the total number of controls.

ROC curves can be generated for a single feature as well as for other single outputs, for example, a combination of two or more features that are combined (such as, added, subtracted, multiplied, weighted, etc.) to provide a single combined value which can be plotted in a ROC curve. The ROC curve is a plot of the true positive rate (sensitivity) of a test against the false positive rate (1-specificity) of the test. ROC curves provide another means to quickly screen a data set.

As used herein “machine learning” refers to algorithms that give a computer the ability to learn without being explicitly programmed including algorithms that learn from and make predictions about data. Machine learning algorithms include, but are not limited to, decision tree learning, artificial neural networks (ANN) (also referred to herein as a “neural net”), deep learning neural network, support vector machines, rule base machine learning, random forest, logistic regression, pattern recognition algorithms, etc. For the purposes of clarity, algorithms such as linear regression or logistic regression can be used as part of a machine learning process. However, it is understood that using linear regression or another algorithm as part of a machine learning process is distinct from performing a statistical analysis such as regression with a spreadsheet program such as Excel. The machine learning process has the ability to continually learn and adjust the classifier model as new data becomes available and does not rely on explicit or rules-based programming. Statistical modeling relies on finding relationships between variables (e.g., mathematical equations) to predict an outcome.

As used herein, the term “increased risk” refers to an increase in the risk level, for a human subject after analysis by the classifier model, for the presence, or development, of a cancer relative to a population's known prevalence of a particular cancer before testing.

Analysis of plasma DNA has enabled novel diagnostic approaches in prenatal(I), transplant(2) and cancer medicine(3). Recent studies have shown fragmentation patterns in cell-free DNA are not random and capture chromatin accessibility in the cells that contribute such DNA into plasma(4). The main source of cell-free DNA in plasma are leukocytes(5). When DNA from white blood cells is shed into plasma, DNA fragments from genomic loci bound by nucleosomes or other DNA binding proteins are protected from degradation(6). Nucleosome positioning and chromatin accessibility across the genome vary between cell types and in different cell states(7). Reflecting this variation, when DNA from a cancer cell is shed into plasma, it may be digested at different genomic loci compared to background DNA from peripheral blood cells. Here, we demonstrate that differences in fragmentation breakpoints of tumor-derived DNA in plasma can serve as a cancer biomarker, using genome wide analysis of positioning and nucleotide sequence at fragment ends in plasma DNA.

### Sample Preparation

The methods of this disclosure may have a wide variety of uses in the manipulation, preparation, identification, quantification and/or analysis of cell free polynucleotides. Examples of polynucleotides include but are not limited to: DNA, RNA, amplicons, cDNA, dsDNA, ssDNA, plasmid DNA, cosmid DNA, high Molecular Weight (MW) DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA (e.g., retroviral RNA).

Cell free polynucleotides may be derived from a variety of sources including human, mammal, non-human mammal, ape, monkey, chimpanzee, reptilian, amphibian, or avian, sources. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant patient) or may be derived from tissue of the patient itself.

Isolation and extraction of cell free polynucleotides may be performed through collection of bodily fluids using a variety of techniques. In some cases, collection may comprise aspiration of a bodily fluid from a patient using a syringe. In other cases, collection may comprise pipetting or direct collection of fluid into a collecting vessel. After collection of bodily fluid, cell free polynucleotides may be isolated and extracted using a variety of techniques known in the art. In some cases, cell free DNA may be isolated, extracted and prepared using commercially available kits such as the Qiagen Qiamp® Circulating Nucleic Acid Kit protocol. In other examples, ThermoFisher MagMAX™ Cell-Free DNA Isolation Kit may be used.

Generally, cell free polynucleotides are extracted and isolated from bodily fluids through a partitioning step in which cell free DNAs, as found in solution, are separated from cells and other non-soluble components of the bodily fluid. Partitioning may include, but is not limited to, techniques such as centrifugation or filtration. In other cases, cells are not partitioned from cell free DNA first, but rather lysed. In this example, the genomic DNA of intact cells is partitioned through selective precipitation. Cell free polynucleotides, including DNA, may remain soluble and may be separated from insoluble genomic DNA and extracted. Generally, after addition of buffers and other wash steps specific to different kits, DNA may be precipitated using isopropanol precipitation. Further clean up steps may be used such as silica-based columns to remove contaminants or salts. General steps may be optimized for specific applications. Nonspecific bulk carrier polynucleotides, for example, may be added throughout the reaction to optimize certain aspects of the procedure such as yield.

Isolation and purification of cell free DNA may be accomplished using any means, including, but not limited to, the use of commercial kits and protocols provided by companies such as Qiagen, ThermoFisher, Sigma Aldrich, Life Technologies, Promega, Affymetrix, P3I or the like. Kits and protocols may also be non-commercially available.

After isolation, in some cases, the cell free polynucleotides are pre-mixed with one or more additional materials, such as one or more reagents (e.g., ligase, protease, polymerase) prior to sequencing.

The methods of this disclosure may also enable the cell free polynucleotides to be tagged or tracked in order to permit subsequent identification and origin of the particular polynucleotide. This feature is in contrast with other methods that use pooled or multiplex reactions and that only provide measurements or analyses as an average of multiple samples. Here, the assignment of an identifier to individual or subgroups of polynucleotides may allow for a unique identity to be assigned to individual sequences or fragments of sequences. This may allow acquisition of data from individual samples and is not limited to averages of samples.

In some examples, nucleic acids or other molecules derived from a single strand may share a common tag or identifier and therefore may be later identified as being derived from that strand. Similarly, all of the fragments from a single strand of nucleic acid may be tagged with the same identifier or tag, thereby permitting subsequent identification of fragments from the parent strand. In still other cases, the systems and methods can be used as a PCR amplification control. In such cases, multiple amplification products from a PCR reaction can be tagged with the same tag or identifier. If the products are later sequenced and demonstrate sequence differences, differences among products with the same identifier can then be attributed to PCR error.

Additionally, individual sequences may be identified based upon characteristics of sequence data for the read themselves. For example, the detection of unique sequence data at the beginning (start) and end (stop) portions of individual sequencing reads may be used, alone or in combination, with the length, or number of base pairs of each sequence read unique sequence to assign unique identities to individual molecules. Fragments from a single strand of nucleic acid, having been assigned a unique identity, may thereby permit subsequent identification of fragments from the parent strand. This can be used in conjunction with bottlenecking the initial starting genetic material to limit diversity.

Further, using unique sequence data at the beginning (start) and end (stop) portions of individual sequencing reads and sequencing read length may be used, alone or combination, with the use of barcodes. In some cases, the barcodes may be unique as described herein. In other cases, the barcodes themselves may not be unique. In this case, the use of non-unique barcodes, in combination with sequence data at the beginning (start) and end (stop) portions of individual sequencing reads and sequencing read length may allow for the assignment of a unique identity to individual sequences. Similarly, fragments from a single strand of nucleic acid having been assigned a unique identity, may thereby permit subsequent identification of fragments from the parent strand.

Generally, the methods and systems provided herein are useful for preparation of cell free polynucleotide sequences to a down-stream application sequencing reaction. Often, a sequencing method is classic Sanger sequencing. Sequencing methods may include, but are not limited to: high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next generation sequencing, Single Molecule Sequencing by Synthesis (SMSS)(Helicos), massively-parallel sequencing, Clonal Single Molecule Array (Solexa), shotgun sequencing. Maxim-Gilbert sequencing, primer walking, and any other sequencing methods known in the art.

### Cancer Detection

The types and number of cancers that detected with the methods disclosed herein include but are not limited to blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like.

In an embodiment, the cancer is selected from the group consisting of oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, glioma, parathyroid cancer, penile cancer, vaginal cancer, thyroid cancer, pancreatic cancer, esophageal cancer, Hodgkin's lymphoma, leukemia-related disorders, mycosis fungoides, hematological cancer, hematological disease, hematological malignancy, minimal residual disease, and myelodysplastic syndrome.

In another embodiment, the cancer is selected from the group consisting of gastrointestinal cancer, prostate cancer, ovarian cancer, breast cancer, head and neck cancer, lung cancer, non small cell lung cancer, cancer of the nervous system, kidney cancer, retina cancer, skin cancer, liver cancer, pancreatic cancer, genital-urinary cancer, colorectal cancer, renal cancer, and bladder cancer.

In another embodiment, the cancer is non-small cell lung cancer, pancreatic cancer, breast cancer, ovarian cancer, colorectal cancer, or head and neck cancer. In yet another embodiment the cancer is a carcinoma, a tumor, a neoplasm, a lymphoma, a melanoma, a glioma, a sarcoma, or a blastoma.

In one embodiment, the carcinoma is selected from the group consisting of carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiated carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial carcinoma, metastatic carcinoma, mucoepidermoid carcinoma, cholangiocarcinoma, actinic keratoses, cystadenoma, and hepatic adenomatosis.

In another embodiment, the tumor is selected from the group consisting of astrocytic tumors, malignant mesothelial tumors, ovarian germ cell tumors, supratentorial primitive neuroectodermal tumors, Wilms tumors, pituitary tumors, extragonadal germ cell tumors, gastrinoma, germ cell tumors, gestational trophoblastic tumors, brain tumors, pineal and supratentorial primitive neuroectodermal tumors, pituitary tumors, somatostatin-secreting tumors, endodermal sinus tumors, carcinoids, central cerebral astrocytoma, glucagonoma, hepatic adenoma, insulinoma, medulloepithelioma, plasmacytoma, vipoma, and pheochromocytoma. In yet another embodiment, the neoplasm is selected from the group consisting of intraepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma. In a further embodiment, the lymphoma may be selected from the group consisting of nervous system lymphoma, AIDS-related lymphoma, cutaneous T-cell lymphoma, non-Hodgkin's lymphoma, lymphoma, and Waldenstrom's macroglobulinemia. In another embodiment, the melanoma may be selected from the group consisting of acral lentiginous melanoma, superficial spreading melanoma, uveal melanoma, lentigo maligna melanomas, melanoma, intraocular melanoma, adenocarcinoma nodular melanoma, and hemangioma. In yet another embodiment, the sarcoma may be selected from the group consisting of adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma. In one embodiment, the glioma may be selected from the group consisting of glioma, brain stem glioma, and hypothalamic and visual pathway glioma. In another embodiment, the blastoma may be selected from the group consisting of pulmonary blastoma, pleuropulmonary blastoma, retinoblastoma, neuroblastoma, medulloblastoma, glioblastoma, and hemangioblastomas.

In certain embodiments, the methods provided herein are used to monitor already known cancers, or other diseases in a particular patient. This allows a practitioner to adapt treatment options in accord with the progress of the disease. In this example, the methods described herein track cfDNA in a particular patient over the course of the disease. In some instances, cancers progress, becoming more aggressive and genetically unstable. In other examples, cancers remain benign, inactive, dormant or in remission. The methods of this disclosure are useful in determining disease progression, remission or recurrence and the appropriate adjustments in treatment that are required for the disease state.

### Cancer Treatment

In certain aspects the disclosed methods further comprise administering at least one treatment to the patient.

A mammal having, or suspected of having, any appropriate type of cancer can be assessed and/or treated using the methods and materials described herein. A cancer can be any stage cancer. In some cases, a cancer can be an early-stage cancer. In some cases, a cancer can be an asymptomatic cancer. In some cases, a cancer can be a residual disease and/or a recurrence (e.g., after surgical resection and/or after cancer therapy).

When treating a mammal having, or suspected of having, cancer as described herein, the mammal can be administered one or more cancer treatments. A cancer treatment can be any appropriate cancer treatment. One or more cancer treatments described herein can be administered to a mammal at any appropriate frequency (e.g., once or multiple times over a period of time ranging from days to weeks). Examples of cancer treatments include, without limitation adjuvant chemotherapy, neoadjuvant chemotherapy, radiation therapy, hormone therapy, cytotoxic therapy, immunotherapy, adoptive T cell therapy (e.g., chimeric antigen receptors and/or T cells having wild-type or modified T cell receptors), targeted therapy such as administration of kinase inhibitors (e.g., kinase inhibitors that target a particular genetic lesion, such as a translocation or mutation), (e.g., a kinase inhibitor, an antibody, a bispecific antibody), signal transduction inhibitors, bispecific antibodies or antibody fragments (e.g., BiTEs), monoclonal antibodies, immune checkpoint inhibitors, surgery (e.g., surgical resection), or any combination of the above. In some cases, a cancer treatment can reduce the severity of the cancer, reduce a symptom of the cancer, and/or to reduce the number of cancer cells present within the mammal.

In some cases, a cancer treatment can include an immune checkpoint inhibitor. Non-limiting examples of immune checkpoint inhibitors include nivolumab (Opdivo), pembrolizumab (Keytruda), atezolizumab (tecentriq), avelumab (bavencio), durvalumab (imfinzi), ipilimumab (vervoy). See, e.g., Pardoll (2012) Nat. Rev Cancer 12: 252-264: Sun et al. (2017) Eur Rev Med Pharmacol Sci 21(6): 1198-1205: Hamanishi et al. (2015) J. Clin. Oncol. 33(34): 4015-22: Brahmer et al. (2012) N Engl J Med 366(26): 2455-65; Ricciuti et al. (2017) J. Thorac Oncol. 12(5): e51-e55: Ellis et al. (2017) Clin Lung Cancer pii: S1525-7304(17)30043-8: Zou and Awad (2017) Ann Oncol 28(4): 685-687: Sorscher (2017) N Engl J Med 376(10: 996-7: Hui et al. (2017) Ann Oncol 28(4): 874-881: Vansteenkiste et al. (2017) Expert Opin Biol Ther 17(6): 781-789; Hellmann et al. (2017) Lancet Oncol. 18(1): 31-41: Chen (2017) J. Chin Med Assoc 80(1): 7-14.

In some cases, a cancer treatment can be an adoptive T cell therapy (e.g., chimeric antigen receptors and/or T cells having wild-type or modified T cell receptors). See, e.g., Rosenberg and Restifo (2015) Science 348(6230): 62-68: Chang and Chen (2017) Trends Mol Med 23(5): 430-450; Yee and Lizee (2016) Cancer J. 23(2): 144-148: Chen et al. (2016) Oncoimmunology 6(2): e1273302: US 2016/0194404; US 2014/0050788: US 2014/0271635; U.S. Pat. No. 9,233,125: incorporated by reference in their entirety herein.

In some cases, a cancer treatment can be a chemotherapeutic agent. Non-limiting examples of chemotherapeutic agents include: amsacrine, azacitidine, axathioprine, bevacizumab (or an antigen-binding fragment thereof), bleomycin, busulfan, carboplatin, capecitabine, chlorambucil, cisplatin, cyclophosphamide, cytarabine, dacarbazine, daunorubicin, docetaxel, doxifluridine, doxorubicin, epirubicin, erlotinib hydrochlorides, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, all-trans retinoic acid, streptozocin, tafluposide, temozolomide, teniposide, tioguanine, topotecan, uramustine, valrubicin, vinblastine, vincristine, vindesine, vinorelbine, and combinations thereof. Additional examples of anti-cancer therapies are known in the art: see. e.g. the guidelines for therapy from the American Society of Clinical Oncology (ASCO). European Society for Medical Oncology (ESMO), or National Comprehensive Cancer Network (NCCN).

When monitoring a mammal having, or suspected of having, cancer as described herein, the monitoring can be before, during, and/or after the course of a cancer treatment. Methods of monitoring provided herein can be used to determine the efficacy of one or more cancer treatments and/or to select a mammal for increased monitoring.

When a mammal is identified as having cancer as described herein, the identifying can be before and/or during the course of a cancer treatment. Methods of identifying a mammal as having cancer provided herein can be used as a first diagnosis to identify the mammal (e.g., as having cancer before any course of treatment) and/or to select the mammal for further diagnostic testing. In some cases, once a mammal has been determined to have cancer, the mammal may be administered further tests and/or selected for further diagnostic testing. In some cases, methods provided herein can be used to select a mammal for further diagnostic testing at a time period prior to the time period when conventional techniques are capable of diagnosing the mammal with an early-stage cancer. For example, methods provided herein for selecting a mammal for further diagnostic testing can be used when a mammal has not been diagnosed with cancer by conventional methods and/or when a mammal is not known to harbor a cancer. In some cases, a mammal selected for further diagnostic testing can be administered a diagnostic test at an increased frequency compared to a mammal that has not been selected for further diagnostic testing. For example, a mammal selected for further diagnostic testing can be administered a diagnostic test at a frequency of twice daily, daily, bi-weekly, weekly, bi-monthly, monthly, quarterly, semi-annually, annually, or any at frequency therein. In some cases, a mammal selected for further diagnostic testing can be administered a one or more additional diagnostic tests compared to a mammal that has not been selected for further diagnostic testing. For example, a mammal selected for further diagnostic testing can be administered two diagnostic tests, whereas a mammal that has not been selected for further diagnostic testing is administered only a single diagnostic test (or no diagnostic tests). In some cases, the diagnostic testing method can determine the presence of the same type of cancer (e.g., having the same tissue or origin) as the cancer that was originally detected. Additionally or alternatively, the diagnostic testing method can determine the presence of a different type of cancer as the cancer that was original detected.

In some cases, the diagnostic testing method is a scan. In some cases, the scan is a computed tomography (CT), a CT angiography (CTA), an esophagram (a Barium swallow), a Barium enema, a magnetic resonance imaging (MRI), a PET scan, an ultrasound (e.g., an endobronchial ultrasound, an endoscopic ultrasound), an X-ray, a DEXA scan. In some cases, the diagnostic testing method is a physical examination, such as an anoscopy, a bronchoscopy (e.g., an autofluorescence bronchoscopy, a white-light bronchoscopy, a navigational bronchoscopy), a colonoscopy, a digital breast tomosynthesis, an endoscopic retrograde cholangiopancreatography (ERCP), an ensophagogastroduodenoscopy, a mammography, a Pap smear, a pelvic exam, a positron emission tomography and computed tomography (PET-CT) scan. In some cases, a mammal that has been selected for further diagnostic testing can also be selected for increased monitoring. Once the presence of a tumor or a cancer (e.g., a cancer cell) has been identified (e.g., by any of the variety of methods disclosed herein), it may be beneficial for the mammal to undergo both increased monitoring (e.g., to assess the progression of the tumor or cancer in the mammal and/or to assess the development of one or more cancer biomarkers such as mutations), and further diagnostic testing (e.g., to determine the size and/or exact location of the tumor or the cancer). In some cases, a cancer treatment is administered to the mammal that is selected for further diagnostic testing after a cancer biomarker is detected and/or after the cfDNA fragmentation profile of the mammal has not improved or deteriorated. Any of the cancer treatments disclosed herein or known in the art can be administered. For example, a mammal that has been selected for further diagnostic testing can be administered a further diagnostic test, and a cancer treatment can be administered if the presence of the tumor or the cancer is confirmed. Additionally or alternatively, a mammal that has been selected for further diagnostic testing can be administered a cancer treatment, and can be further monitored as the cancer treatment progresses. In some cases, after a mammal that has been selected for further diagnostic testing has been administered a cancer treatment, the additional testing will reveal one or more cancer biomarkers (e.g., mutations). In some cases, such one or more cancer biomarkers (e.g., mutations) will provide cause to administer a different cancer treatment (e.g., a resistance mutation may arise in a cancer cell during the cancer treatment, which cancer cell harboring the resistance mutation is resistant to the original cancer treatment).

### Machine Learning

In the present disclosure, the classifier models are “trained” using machine learning systems by building a model from inputs. Those inputs may be longitudinal data, wherein a known diagnosis of cancer (including matched controls) is determined months, if not years, after data from measured biomarkers and clinical factors of those patients is collected.

In certain aspects, the methods include a first classifier model, generated by a machine learning system, that classifies a patient into a risk category of having or developing cancer.

In some aspects, use of the classifier model assigns a risk score of having or developing cancer to the patient using input variables of age and the measured values of biomarkers from the patient when an output of the classifier model is a numerical expression of the percent likelihood of having or developing cancer. In embodiments, the classifier model classifies a patent into a risk category of having or developing cancer using the assigned risk score, wherein a risk score percent likelihood of having or developing cancer is greater than the percent prevalence of cancer in the population is deemed an increased risk category. As used herein, the term “increased risk” refers to an increase for the presence, or development, of the cancer as compared to the known prevalence of that particular cancer across the population cohort. The known prevalence of cancer is typically between 0.5 and 3% in a population.

In certain embodiments the classifier model is static, and its use is implemented by a computer-implemented system comprising at least one processor and at least one memory, the at least one memory comprising instructions executed by the at least one processor to cause the at least one processor to implement the classifier model. In certain embodiments, a machine learning system iteratively regenerates the classifier model by training the classifier model with new training data to improve the performance of the classifier model. The first classifier model yields a numerical risk score for each patient tested, which can be used by physicians to further inform screening procedures to better predict and diagnose early stage cancer in asymptomatic patients. Also, as disclosed in more detail herein, the machine learning system is adapted to receive additional data as the system is used in a real-world clinical setting and to recalculate and improve the performance so that the classifier model becomes “smarter” the more it is used.

Any machine learning algorithm may be used to analyze the data including, for example, a random forest, a support vector machine (SVM), or a boosting algorithm (e.g., adaptive boosting (AdaBoost), gradient boost method (GBM), or extreme gradient boost methods (XGBoost)), or neural networks such as H20.

Machine learning algorithms generally are of one of the following types: (1) bagging (decrease variance), (2) boosting (decrease bias), or (3) stacking (improving predictive force). In bagging, multiple prediction models (generally of the same type) are constructed from subsets of classification data (classes and features) and then combined into a single classifier. Random Forest classifiers are of this type. In boosting, an initial prediction model is iteratively improved by examining prediction errors. AdaBoost and extreme Gradient Boosting are of this type. In stacking models, multiple prediction models (generally of different types) are combined to form the final classifier. These methods are called ensemble methods. The fundamental or starting methods in the ensemble methods are often decision trees. Decision trees are non-parametric supervised learning methods that use simple decision rules to infer the classification from the features in the data. They have some advantages in that they are simple to understand and can be visualized as a tree starting at the root (usually a single node) and repeatedly branch to the leaves (multiple nodes) that are associated with the classification.

In some embodiments, methods of the disclosure use a machine learning system that uses a random forest. Random forests use decision tree learning, where a model is built that predicts the value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, 2001, Random Forests, Machine Learning 45:5-32, incorporated by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable.

SVMs can be used for classification and regression. When used for classification of new data into one of two categories, such as having a disease or not having a disease, a SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines. Numerical Recipes: The Art of Scientific Computing (3rd ed.). New York: Cambridge University (2007), incorporated herein by reference. SVMs can also be used in support vector clustering. See Ben-Hur, 2001, Support Vector Clustering. J Mach Learning Res 2:125-137, incorporated by reference.

Boosting algorithms are machine learning ensemble meta-algorithms for reducing bias and variance. Boosting is focused on turning weak learners into strong learners where a weak learner is defined to be a classifier which is only slightly correlated with the true classification while a strong learner is a classifier that is well-correlated with the true classification. Boosting algorithms consist of iteratively learning weak classifiers with respect to a distribution and adding them to a final strong classifier. The added classifiers are typically weighted in based on their accuracy. Boosting algorithms include AdaBoost, gradient boosting, and XGBoost. See Freund, 1997, A decision-theoretic generalization of on-line learning and an application to boosting. J Comp Sys Sci 55:119; and Chen, 2016, XGBoost: A Scalable Tree Boosting System, arXiv: 1603.02754, both incorporated by reference.

Neural networks, modeled on the human brain, allow for processing of information and machine learning. Neural networks include nodes that mimic the function of individual neurons, and the nodes are organized into layers. Neural networks include an input layer, an output layer, and one or more hidden layers that define connections from the input layer to the output layer. Systems and methods of the invention may include any neural network that facilitates machine learning. The system may include a known neural network architecture, such as GoogLeNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012): VGG16 (Simonyan & Zisserman, Very deep convolutional networks for large-scale image recognition, CoRR, abs/3409.1556, 2014); or FaceNet (Wang et al., Face Search at Scale: 80 Million Gallery, 2015), each of the aforementioned references are incorporated by reference. Deep learning neural networks (also known as deep structured learning, hierarchical learning or deep machine learning) include a class of machine learning operations that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower level features to form a hierarchical representation. Those features are preferably represented within nodes as feature vectors. Deep learning by the neural network includes learning multiple levels of representations that correspond to different levels of abstraction; the levels form a hierarchy of concepts. In some embodiments, the neural network includes at least 5 and preferably more than ten hidden layers. The many layers between the input and the output allow the system to operate via multiple processing layers.

Deep learning is part of a broader family of machine learning methods based on learning representations of data. An observation can be represented in many ways such as a vector of intensity values per pixel, or in a more abstract way as a set of edges, regions of particular shape, etc. Those features are represented at nodes in the network. Preferably, each feature is structured as a feature vector, a multi-dimensional vector of numerical features that represent some object. The feature provides a numerical representation of objects, since such representations facilitate processing and statistical analysis. Feature vectors are similar to the vectors of explanatory variables used in statistical procedures such as linear regression. Feature vectors are often combined with weights using a dot product in order to construct a linear predictor function that is used to determine a score for making a prediction.

The vector space associated with those vectors may be referred to as the feature space. In order to reduce the dimensionality of the feature space, dimensionality reduction may be employed. Higher-level features can be obtained from already available features and added to the feature vector, in a process referred to as feature construction. Feature construction is the application of a set of constructive operators to a set of existing features resulting in construction of new features.

Within the network, nodes are connected in layers, and signals travel from the input layer to the output layer. In certain embodiments, each node in the input layer corresponds to a respective one of the features from the training data. The nodes of the hidden layer are calculated as a function of a bias term and a weighted sum of the nodes of the input layer, where a respective weight is assigned to each connection between a node of the input layer and a node in the hidden layer. The bias term and the weights between the input layer and the hidden layer are learned autonomously in the training of the neural network. The network may include thousands or millions of nodes and connections. Typically, the signals and state of artificial neurons are real numbers, typically between 0 and 1. Optionally, there may be a threshold function or limiting function on each connection and on the unit itself, such that the signal must surpass the limit before propagating. Back propagation is the use of forward stimulation to modify connection weights and is sometimes done to train the network using known correct outputs. See WO 2016/182551, U.S. Pub. 2016/0174902, U.S. Pat. No. 8,639,043, and U.S. Pub. 2017/0053398, each incorporated by reference.

In some embodiments, the datasets are used to cluster a training set. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Bayesian networks are probabilistic graphical models that represent a set of random variables and their conditional dependencies via directed acyclic graphs (DAGs). The DAGs have nodes that represent random variables that may be observable quantities, latent variables, unknown parameters or hypotheses. Edges represent conditional dependencies: nodes that are not connected represent variables that are conditionally independent of each other. Each node is associated with a probability function that takes, as input, a particular set of values for the node's parent variables and gives (as output) the probability (or probability distribution, if applicable) of the variable represented by the node.

Regression analysis is a statistical process for estimating the relationships among variables such as features and outcomes. It includes techniques for modeling and analyzing relationships between a multiple variable. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning.

In some embodiments, the machine learning system may learn in a supervised or unsupervised fashion. A machine learning system that learns in an unsupervised fashion may be referred to as an autonomous machine learning system. While other versions are within the scope of the invention, an autonomous machine learning system can employ periods of both supervised and unsupervised learning. As such, in one embodiment, the random forest may be operated autonomously and may include periods of both supervised and unsupervised learning. See Criminisi, 2012, Decision Forests: A unified framework for classification, regression, density estimation, manifold learning and semi-supervised learning, Foundations and Trends in Computer Graphics and Vision 7(2-3):81-227, incorporated by reference. Thus, in some embodiments, an autonomous machine learning system comprises a random forest. In some embodiments, the autonomous machine learning system discovers the associations via operations that include at least a period of unsupervised learning.

Where the algorithm is trained on treatment outcomes, it can then be used to predict a patient's responsiveness to various cancer-specific therapies. Accordingly, methods may include recommending a treatment based in part on the prediction where a certain treatment will only be recommended for patients likely to respond thereto. In certain embodiments, the recommended treatment may be provided in a report for the patient or a treating physician. In some embodiments, the treatment may be prescribed for the patient or administered to the patient.

The method disclosed herein may be provided with patient data from an individual. That is, the machine learning system has learned from the training data set patterns or associations that are predictive of disease. The system may then be applied to an individual to predicting a cancer state for the individual when the patient data presents one or more of the discovered associations. Upon detecting that association among the patient data for the individual, the machine learning system further generates a report providing information related to the cancer evaluation.

In some aspects, a machine learning model is used for detection of disease. The output of a machine learning model can be the probability that the tested sample is from a cancer patient. ROC curves are developed using different thresholds of this probability. In certain aspects, the machine learning model is trained on a representative set of case and control samples (e.g., samples from cancer patients and healthy patients). A finalized random forest model can be used to generate probability of disease (e.g., cancer) for each new test sample from a patient. The probabilities can be reported as an output. Alternative, based on pre-established thresholds for probability, detection of cancer can be determined and reported as an output. If cancer is detected, the patients may then undergo further clinical and radiological evaluation.

In other aspects, the machine learning classifier is configured to compute a probability of presence of disease, at least in part, on the fraction of aberrant fragments (FAF) and/or average nucleotide frequencies at start sites and end sites of cfDNA fragments. In one embodiment, the computed probability is within the range [0, 1]. In one embodiment, the machine learning classifier is a quadratic discriminant analysis (QDA) classifier. In another embodiment, the machine learning classifier may be another, different type of machine learning classifier, for example, a linear discriminant analysis (LDA) classifier, a support vector machine (SVM) classifier, a random forests (RF) classifier, or a deep-learning classifier, including a convolutional neural network (CNN), configured to compute a probability of presence of disease based, at least in part, on the fraction of aberrant fragments (FAF) and/or average nucleotide frequencies at start sites and end sites of cfDNA fragments. Providing the fraction of aberrant fragments (FAF) and/or average nucleotide frequencies at start sites and end sites of cfDNA fragments to the machine learning classifier may include acquiring electronic data, reading from a computer file, receiving a computer file, reading from a computer memory, or other computerized activity not practically performed in the human mind.

The machine learning classifier may compute the probability based, at least in part, on the fraction of aberrant fragments (FAF) and/or average nucleotide frequencies at start sites and end sites of cfDNA fragments. In various embodiments, the probability can comprise one or more of a most likely diagnosis, for example, as determined based on the fraction of aberrant fragments (FAF) and/or average nucleotide frequencies at start sites and end sites of cfDNA fragments, a probability or confidence associated with a most likely diagnosis. Receiving the probability from the machine learning classifier may include acquiring electronic data, reading from a computer file, receiving a computer file, reading from a computer memory, or other computerized activity not practically performed in the human mind.

In an embodiment of the present invention, a program code implementing the disclosed methods may use a binning procedure using the average value of the corresponding feature as threshold, for example, values above the threshold are coded as 1, and values below it as 0.

In an embodiment of the present invention, the program code utilizes the pre-processed data or access available data sets to build a training set by using statistical sampling. The training set includes data representing the event and data that represent an absence of the event. In some embodiments of the present invention, the training set comprises electronic records that are only readable by a computing resource.

In other embodiments, the program code formulates the training set by proportionally selecting representative electronic records from the target and control populations: the target population is the population with the condition (e.g., event, disease) and the control population is the population is the negative case (to distinguish from the target). Thus, in the example where an event is a disease, the training set includes disease entries and healthy entries. Departing from the specific disease example, in an embodiment of the present invention, the program code utilizes a test set of training data to train the machine learning algorithm. The training set is selected to include both records with the occurrence or condition the algorithm was generated to identify, and records absent this occurrence or condition. The program code tests/trains the individual features that comprise the mutual information (and/or other technologies discussed herein) selected to identify a given condition, and utilizing voting and ensemble learning, trains the algorithm.

In an embodiment of the present invention, the program code may utilize the training set with the significant patterns identified in the analysis to construct and tune a machine learning algorithm, such that the algorithm can distinguish data comprising the event from data that does not comprise the event. The machine learning algorithm may be a linear SVM classification algorithm, which can be utilized with one or more of an RF grouping algorithm and/or a log regression. If the event is a disease, including a cancer, the program code may train the machine learning algorithm to separate database entries representing individuals with a disease from entries representing healthy individuals and/or individuals without this particular disease. The program code may utilize the machine learning algorithm, may assign probabilities to various records in the data set during training runs and the program code, may continue training the algorithm until the probabilities accurately reflect the presence and/or absence of a condition in the records within a pre-defined accuracy threshold. With certain diseases, the program code utilizes a support vector machine (SVM) classifier. The program code makes a selection based on a comparative assessment of various classifiers. When building a model for a cancer, in some embodiments of the present invention, the program code utilizes random forest to generate predictors.

In some embodiments of the present invention, using the disease example, the training set represents a patient population that had the disease. The machine learning algorithm, which is discussed herein, learns from this defined patient population. In essence, the machine learning algorithm uses a surrogate patient population to find the undiagnosed patients. Stated in another way, the surrogate patient population consists of the patients known to have the disease, and the machine learning algorithms encode their pre-diagnosis characteristics to find similar patients and process the retrospective patient journey to predict the prospective patient journey. In the patient definition process the program code identifies cohort of patients that the machine learning algorithm will learn from: this patient cohort will serve as the training set. In embodiments of the present invention, the internal algorithms applied by the program code include, but are not limited to: 1) mutual information to inform or refine the patient definition; and/or 2) various data mining techniques, including but not limited to, histograms to capture various types of data including geographic location, patient demographics (age, gender), and co-morbidities.

In other aspects, the program code constructs the machine learning algorithm, which can be understood as a classifier, as it classifies records (which may represent individuals) into a group with a given condition and a group without the given condition. In an embodiment of the present invention, the program code utilizes the frequency of occurrences of features in the mutual information to identify and filter out false positives. The program code utilizes the classifier to create a boundary between individuals with a condition and the general population to lower multi-dimensional planes, given multiple dimensions, including, for example, fifty (50) to one hundred (100) dimensions.

As part of constructing a classifier (machine learning algorithm), the program code may test the classifier to tune its accuracy. In an embodiment of the present invention, the program code feeds the previously identified feature set into a classifier and utilizes the classifier to classify records of individuals based on the presence or absence of a given condition, which is known before the tuning. As aforementioned, the presence or absence of the condition is not noted explicitly in the records of the data set. When classifying an individual with a given condition utilizing the classifier, the program code may indicate a probability of a given condition with a rating on a scale, for example, between 0 and 1, where 1 would indicate a definitive presence. The classifier may also exclude certain individuals, based on the medical data of the individual, from the condition.

In an embodiment of the present invention, the program code constructs more than one machine learning algorithm, each with different parameters for classification, based on different analysis of the mutual information, and generates an ultimate machine learning algorithm based on a sum of these classifiers.

In an embodiment of the present invention, to decrease the instances of false positive results, in an embodiment of the present invention, when the algorithm is an SVM algorithm, the program code collects false positive results and sorts them according to their SVM score to identify false positives. In an embodiment of the present invention, to increase the comprehensibility and usability of the result, the program code post-processes records identified as including the event according to pre-defined logical filters. These pre-defined filters may be clinically derived.

All headings are for the convenience of the reader and should not be used to limit the meaning of the text that follows the heading, unless so specified.

The present invention is further illustrated by the following examples that should not be construed as limiting. The contents of all references, patents, and published patent applications cited throughout this application, as well as the Figures, are incorporated herein by reference in their entirety for all purposes.

### Examples

### Example 1. Experimental Methods

### Patients and Samples

Blood samples from patients with breast cancer were collected from patients with glioblastoma, and from patients with cholangiocarcinoma. For a subset of patients with cancer, multiple blood samples were collected including at presentation and during treatment.

### Sample Processing, DNA Extraction and Sequencing

Blood samples were collected in EDTA BD Vacutainer tubes. Plasma was separated within 3 hours of venipuncture by centrifugation at 820 g for 10 minutes, followed by a second centrifugation at 16000 g for 10 minutes. One milliliter aliquots of plasma were stored at −80° C. until DNA extraction. DNA was extracted using either MagMAX Cell-Free DNA Isolation Kit (ThermoFisher) or QIAamp Circulating Nucleic Acid Kit (Qiagen) from 1 ml to 4 ml plasma. Cell-free DNA was quantified prior to library preparation using Qubit dsDNA HS assay (ThermoFisher), Cell-free DNA ScreenTape on the TapeStation 4200 (Agilent), or using an in-house digital PCR assay(21). Whole genome sequencing libraries were prepared from plasma DNA using ThruPLEX Plasma-Seq or Tag-seq (Takara). Libraries were sequenced on HiSeq 4000, NextSeq 550, or NovaSeq 6000 (Illumina) to generate 75 bp to 150 bp paired-end reads.

### Sequencing Data Analysis

Sequencing data was converted to fastq files using bcl2fastq v2.20.0.422. Sequencing reads were trimmed using fastp v0.20.0(22). Trimmed reads were aligned to human genome build hs37d5 (hg19) using bwa-mem v0.7.16a(23) and converted to bam files using samtools 1.9-92-gcb6b3b5(24). Tumor fraction was inferred using copy number analysis of plasma DNA using ichorCNA v0.3.2, together with hmmcopy for patients with melanoma and cholangiocarcinoma(25, 26). Reported limit of detection using ichorCNA is 3% tumor fraction. Any samples non-detectable using ichorCNA were incorporated as zeros in correlation analyses.

### External Data

Fragment end positions, and clinical annotation for patients with cancer and healthy individuals from three published studies(25, 27, 28) were obtained from FinaleDB(29). These data were processed similarly to fragment end positions identified from patients in this study.

### Analysis of Fragment Ends

To analyze genomic positioning of fragment ends, a map of recurrently protected regions was inferred from 17 heathy individuals (sequenced to ˜30× coverage each), using a peak-calling method based on window-protection scores (30). Using this map, cell-free fragments were identified as aberrant if one or both of ends were located within a protected region. Non-aberrant fragments were identified as those that span the length of a protected region. Using the counts of these two types of fragments, fraction of aberrant fragments (FAF) was calculated as the ratio of aberrant fragments to the total number of aberrant and non-aberrant fragments.

To analyze average nucleotide frequencies at fragment ends, positions from 10 bp upstream to 10 bp downstream of each fragment end were considered. For each plasma sample, average frequency across all fragments was calculated for each combination of position and base, using the sequence represented in the hg19 reference genome. Mono-nucleotide frequency was calculated at each position using samtools(24), BEDTools v2.29.0(31) and homerTools v4.11 (32). Each sample was represented by a vector of 168 length (2 fragment ends×4 bases×21 positions).

For building the classification model, we used the nucleotide frequency vector and FAF for each sample. Samples were stratified by cancer type (single stratification for healthy) and split into 80% train and 20% test data. Such stratified splits ensured that train and test data share similar representation of different types of data variations, leading to improved generalization on test data (33). A random forest classifier (using 100 decision trees) was trained and evaluated over 100 runs and using 1000 activation thresholds uniformly distributed between 0 and 1. This binary classifier was trained using a label of 0 for healthy samples and 1 for samples from patients with cancer. The data used for building this model was limited to one sample per patient (the earliest time point available for each), to avoid potential signal leakage between train and test data.

### Down-Sampling Analysis

To evaluate whether analysis of fragment ends was robust at lower read depths, the original datasets were subsampled using samtools (24). To calculate coefficient of variation for FAF, subsampling analysis was performed using earliest available plasma samples from 35 patients with melanoma. The full dataset was randomly subsampled 10 times with a maximum of 1 to 10 million fragments. With FAF computed for each random sample, coefficient of variation was calculated per observation for a given number of reads. To assess how our robust classification model was at lower read depths, a random subsample of 1 to 10 million reads was obtained from each sample included in the model and including in training and evaluation.

### Comparison of FAF Across Copy Number Aberrations

To compare differences in FAF between genomics regions affected by copy number aberrations, 27 plasma samples from patients with melanoma with tumor fractions of at least 20% were selected. FAF was calculated in non-overlapping 500 kb windows across the genome in each sample, along with 24 healthy control samples. For each plasma sample, we identified all windows that completely overlapped with copy number segments having less than, equal to, or greater than 2 copies. For each window, we calculated the z-score of the patient sample versus healthy controls by subtracting the mean FAF value of the bin in the healthy samples from the patient sample and dividing by the standard deviation of the healthy sample FAF values.

### Comparison of FAF for Mutated and Non-Mutated Fragments

To compare FAF between mutated and non-mutated fragments, tumor and germline exome sequencing data from two patients with metastatic melanoma were analyzed, as described in an earlier study (19). Deep whole genome sequencing of the corresponding plasma samples was performed. Genomic loci where mutations were identified in the tumor DNA were interrogated in corresponding plasma WGS data. FAF was calculated for mutated and non-mutated fragments, in aggregate for all mutations.

### Targeted Digital Sequencing of Plasma DNA

Tumor fraction in plasma samples from patients with glioblastoma was measured using targeted digital sequencing as described earlier (34). Briefly, patient-specific somatic mutations were selected by analyzing exome sequencing data from tumor biopsies and germline DNA. Clonal mutations were identified, adjusting for copy number aberrations in the tumor genome and overall tumor purity. Target-specific multiplexed primers were designed and evaluated for in vitro performance using control DNA samples. Sequencing libraries were prepared and sequenced on an Illumina NovaSeq S4 flow cell. Sequencing data were analyzed to evaluate targeted genomic loci and determine confidence in ctDNA detection in each sample. ctDNA fraction was calculated as the mean of all measured variant allele fractions.

### Statistical Analysis

Statistical analyses were performed using Julia and Python. Significance values of differences between two FAF distributions were evaluated using the t test. Statistical significance between distribution of FAF in copy number loss, neutral, or gain regions was calculated using the Mann-Whitney U test. To compute the statistical significance of correlation, the correlation values were first converted to a t statistic and then converted to a P value based on population size. Comparison of FAF between mutated and non-mutated DNA fragments within a plasma sample was performed using the two-proportions Z test. All P values reported are two-sided. P values smaller than 0.05 were considered statistically significant.

### Example 2. Determination of Genomic Regions Protected from Degradation and Analysis of Aberrant cfDNA Fragments

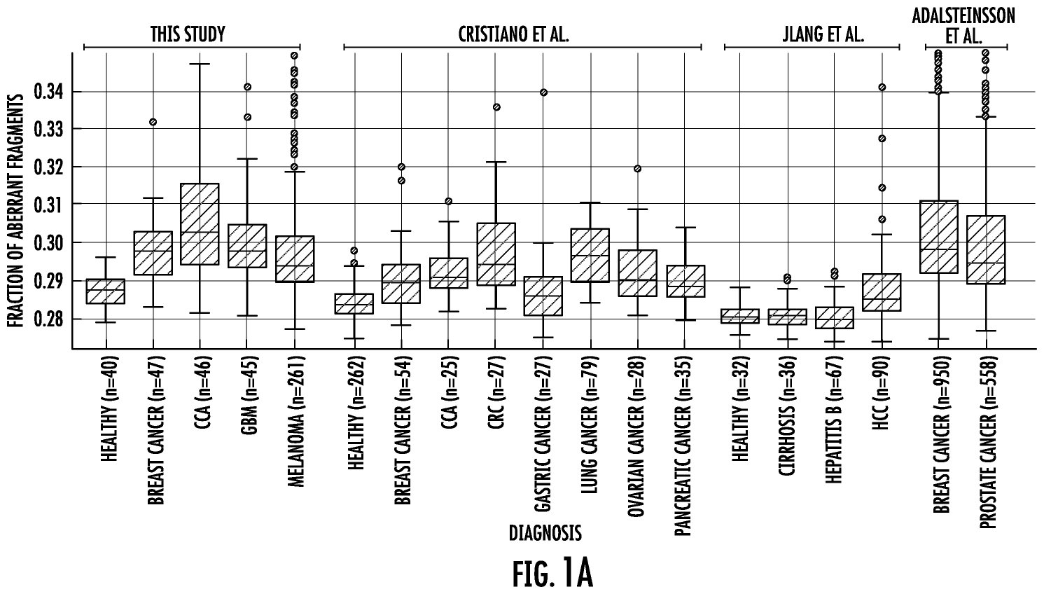

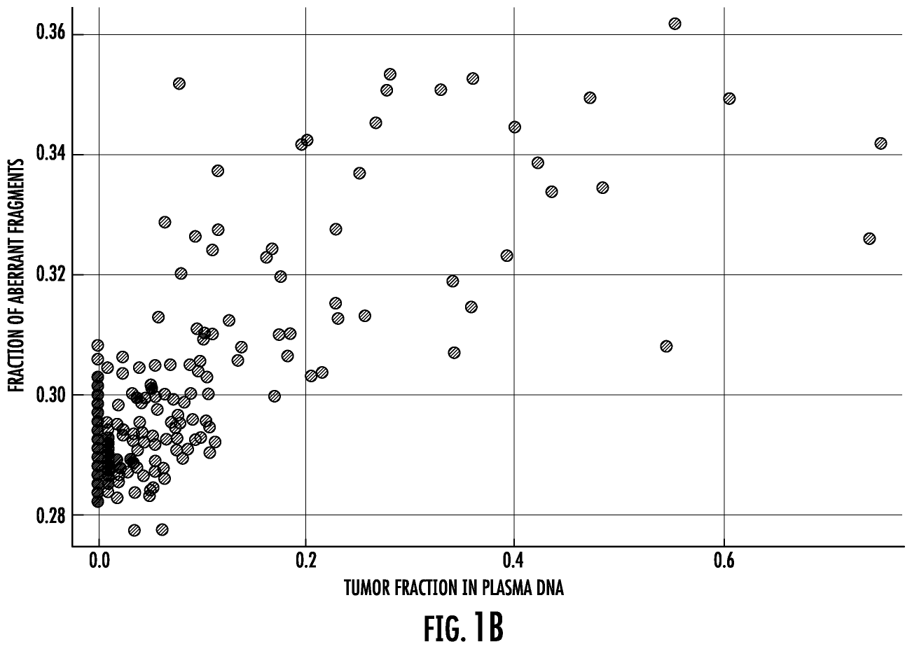

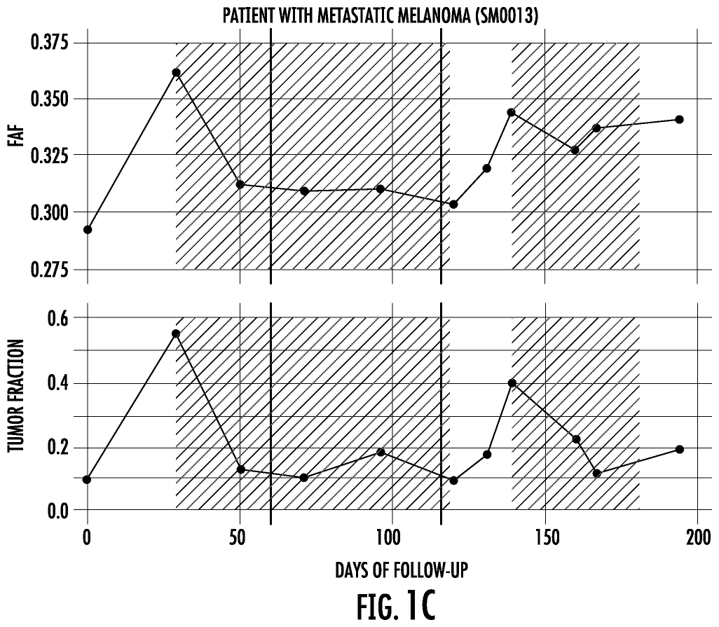

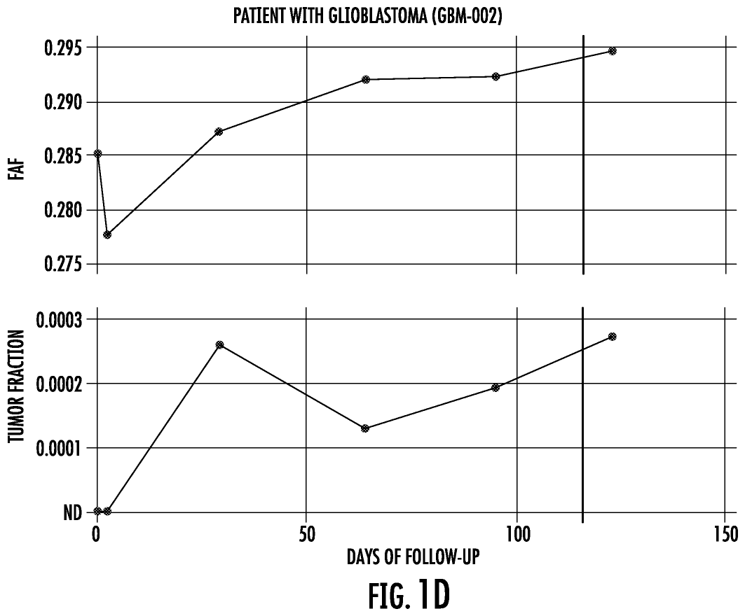

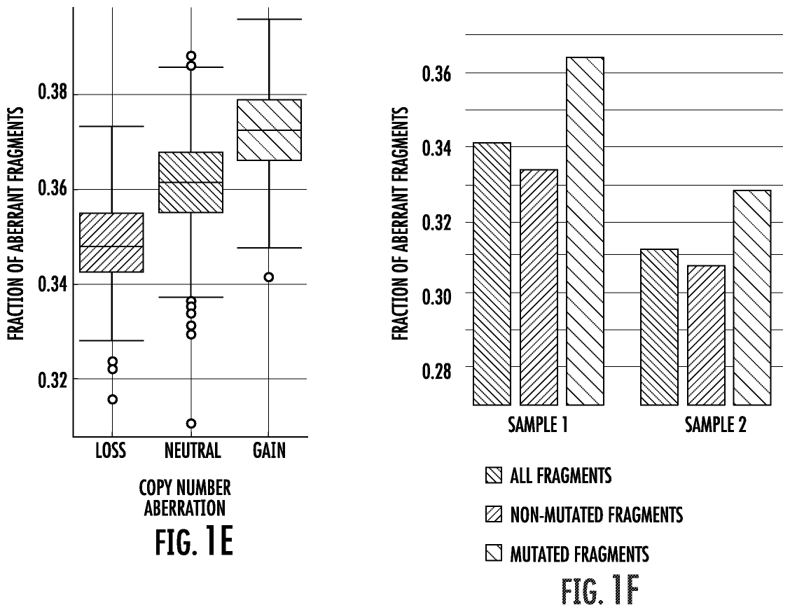

To evaluate whether genomic positioning of fragment ends in plasma DNA was different between cancer patients and healthy individuals, we first inferred a map of genomic regions recurrently protected from degradation using whole genome sequencing of plasma DNA from 17 healthy individuals. Using this map, we quantified the fraction of aberrant fragments that have fragment ends within recurrently protected regions in plasma samples from cancer patients and healthy individuals (FIG. 1A and TABLE 1).

TABLE 1

| | | Number | | Standard | |
|---|---|---|---|---|---|
| | | of | Mean | Deviation | |
| Study | Diagnosis | Samples | FAF | of FAF | P value |
| This study | Healthy Individuals | 40 | 0.287 | 0.004 | — |
| | Breast Cancer | 47 | 0.299 | 0.012 | 4.5 × 10⁻⁸ |
| | Cholangiocarcinoma | 46 | 0.307 | 0.016 | 9.4 × 10⁻¹¹ |
| | Glioblastoma | 45 | 0.301 | 0.013 | 9.3 × 10⁻⁹ |
| | Melanoma | 261 | 0.299 | 0.016 | 3.3 × 10⁻⁶ |
| Cristiano | Healthy Individuals | 262 | 0.285 | 0.005 | — |
| et al. (27) | Breast Cancer | 54 | 0.290 | 0.008 | 6.9 × 10⁻⁹ |
| | Cholangiocarcinoma | 25 | 0.293 | 0.006 | 4.7 × 10⁻¹¹ |
| | Colorectal Cancer | 27 | 0.298 | 0.014 | 4.7 × 10⁻²¹ |
| | Gastric Cancer | 27 | 0.288 | 0.012 | 8.2 × 10⁻³ |
| | Lung Cancer | 79 | 0.293 | 0.009 | 1.5 × 10⁻²¹ |
| | Ovarian Cancer | 28 | 0.292 | 0.009 | 1.4 × 10⁻¹⁰ |
| | Pancreatic Cancer | 35 | 0.290 | 0.007 | 8.0 × 10⁻⁸ |
| Jiang | Healthy Individuals | 32 | 0.281 | 0.003 | — |
| et al. (28) | Liver Cirrhosis | 36 | 0.281 | 0.004 | 0.65 |
| | Hepatitis B | 67 | 0.280 | 0.004 | 0.52 |
| | Hepatocellular | 90 | 0.288 | 0.010 | 1.5 × 10⁻⁴ |
| | Carcinoma | | | | |
| Adalsteinsson | Breast Cancer | 950 | 0.304 | 0.019 | 1.5 × 10⁻⁸ |
| et al. (25) | Prostate Cancer | 558 | 0.301 | 0.017 | 9.7 × 10⁻⁷ |

TABLE 1 shows a comparison of FAF between analyzed samples and cohorts. For each study, groups of patients were compared with data from the study's corresponding healthy individual samples. For Adalsteinsson et al., no healthy individual sample data was available and patient groups were compared with healthy individuals in our study. Two-tailed p values are reported from Student's t-test. No significant elevation in FAF was observed for patients with liver cirrhosis or hepatitis B.

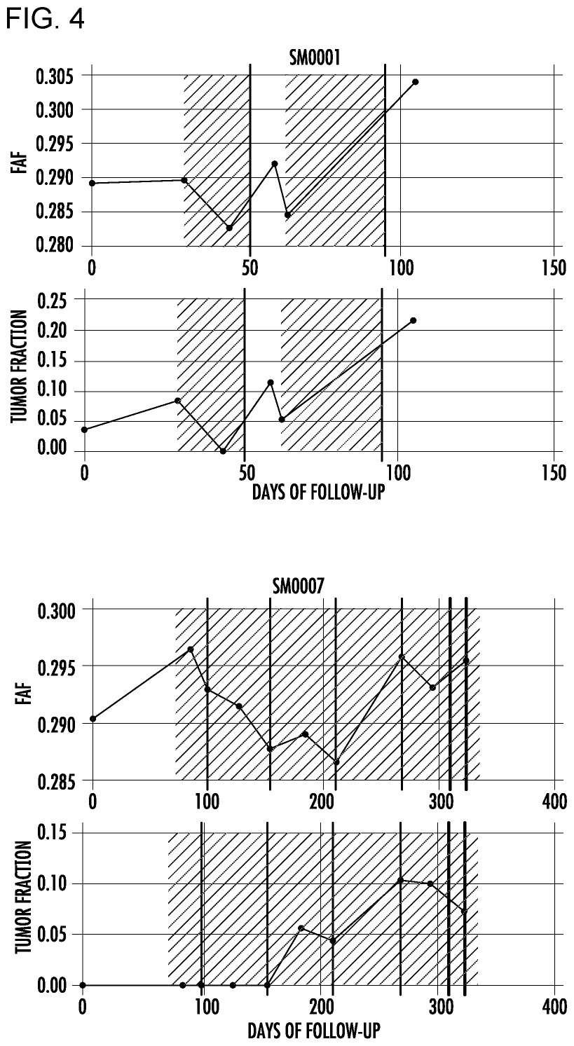

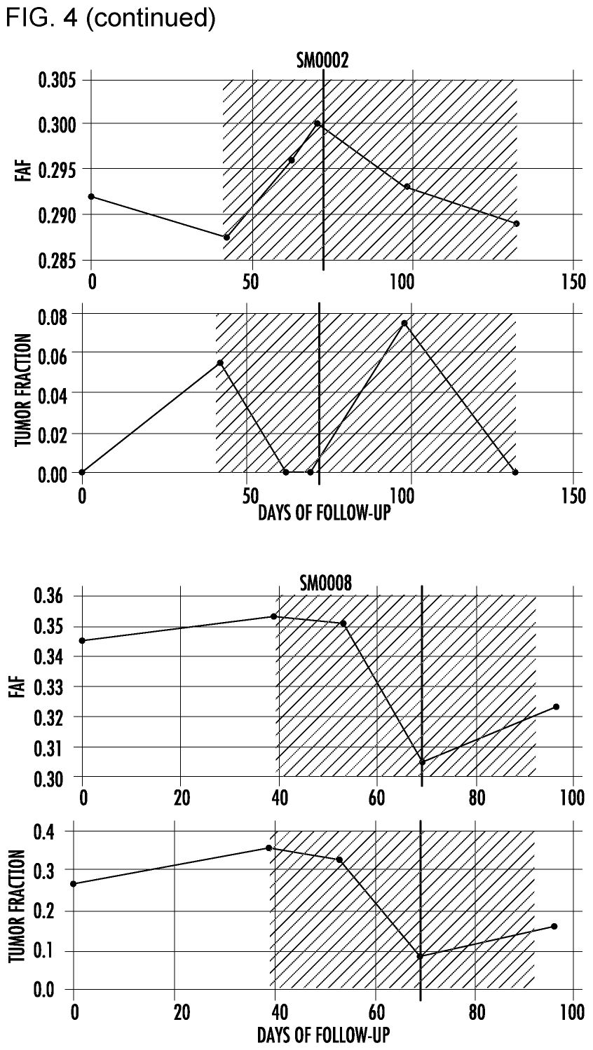

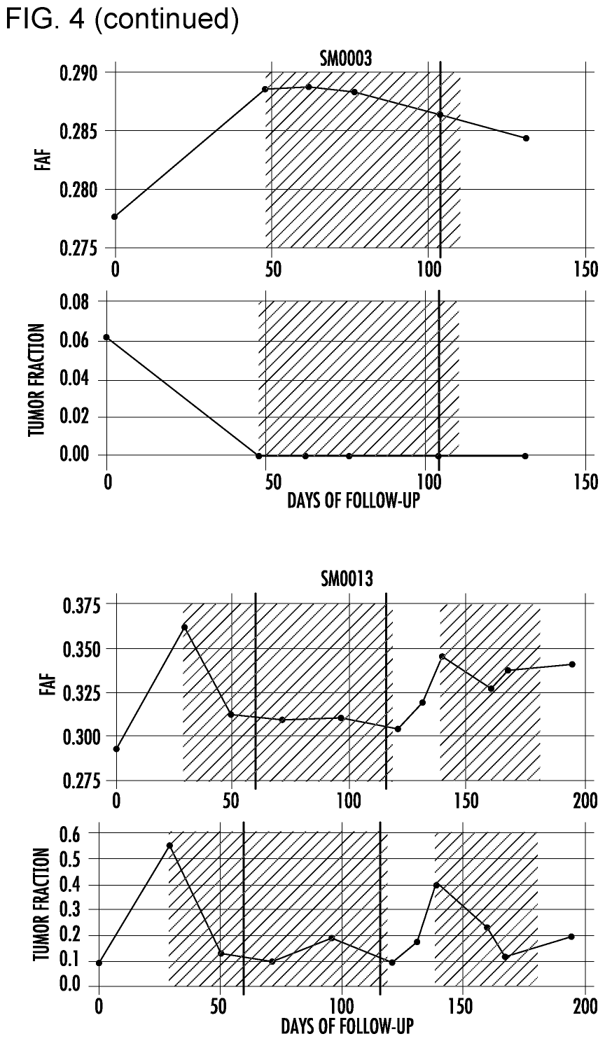

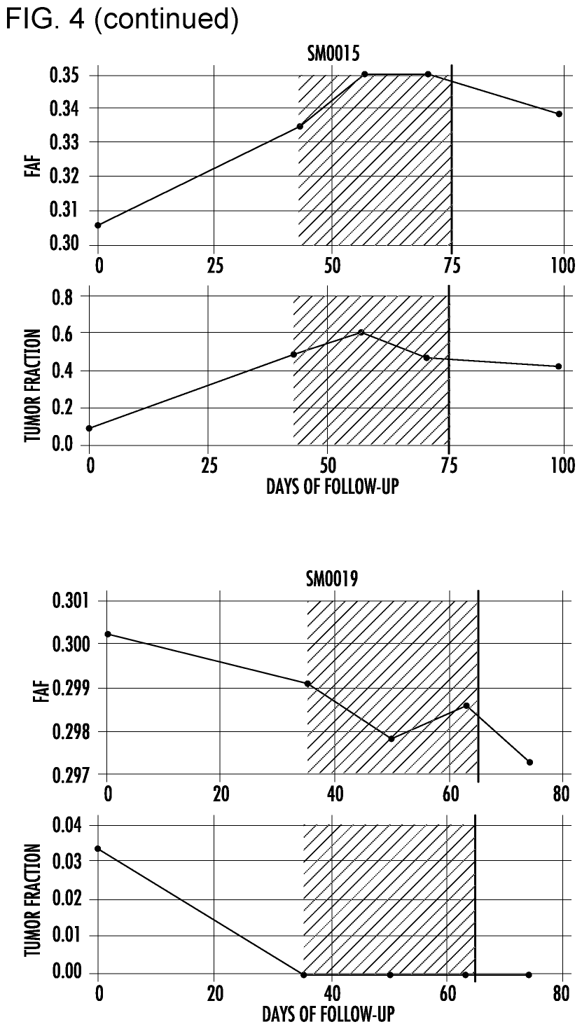

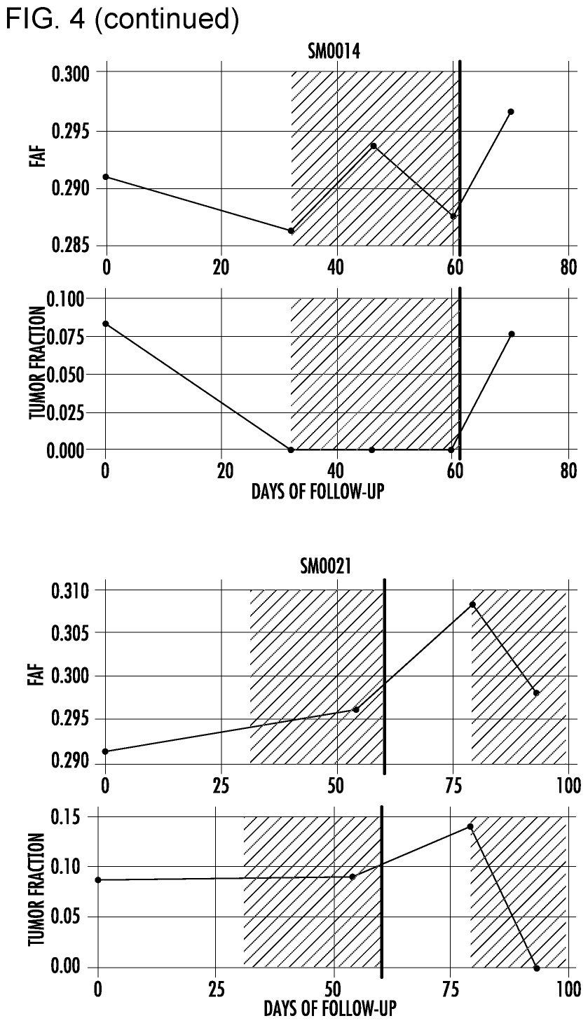

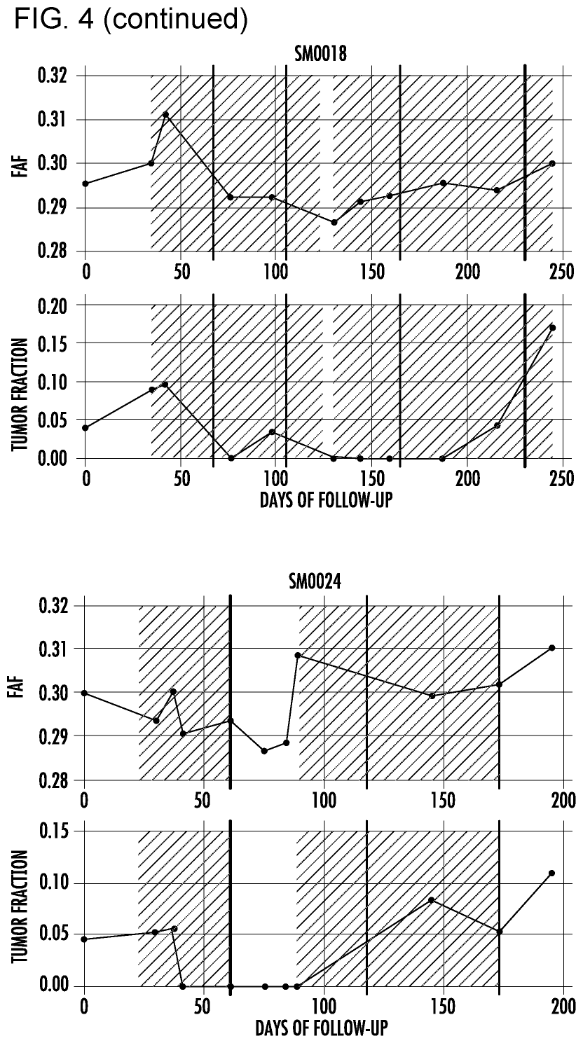

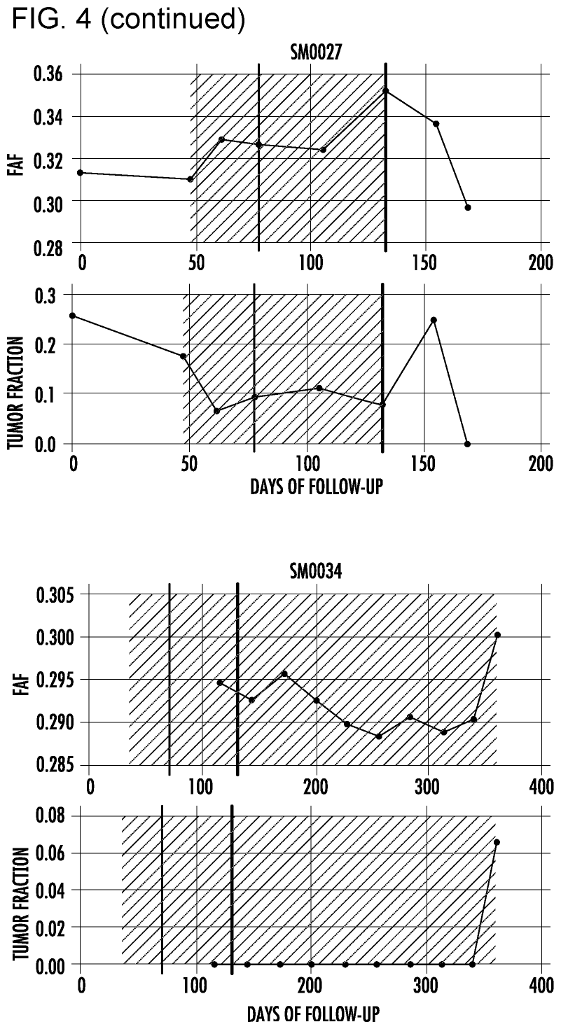

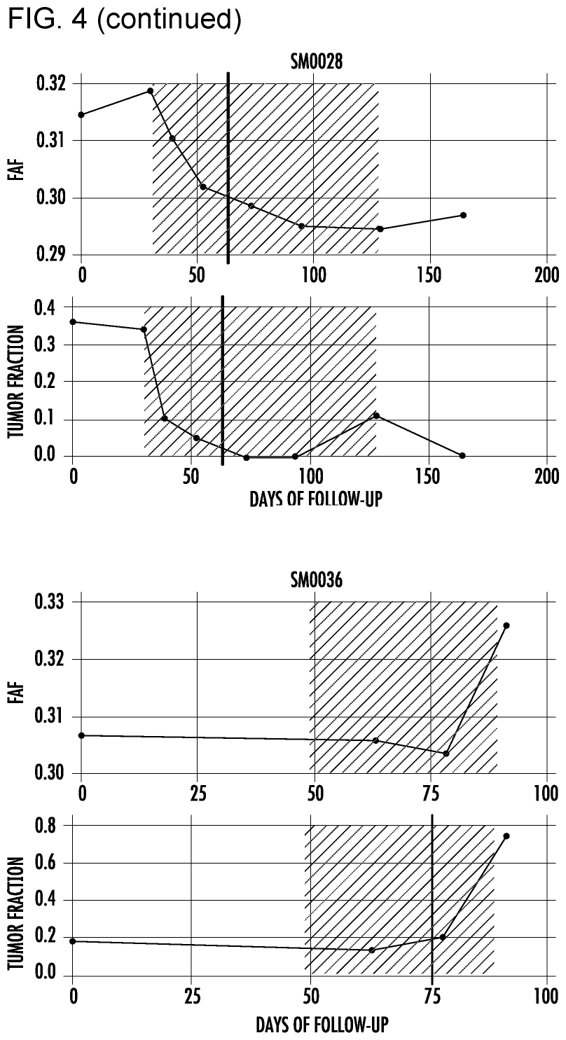

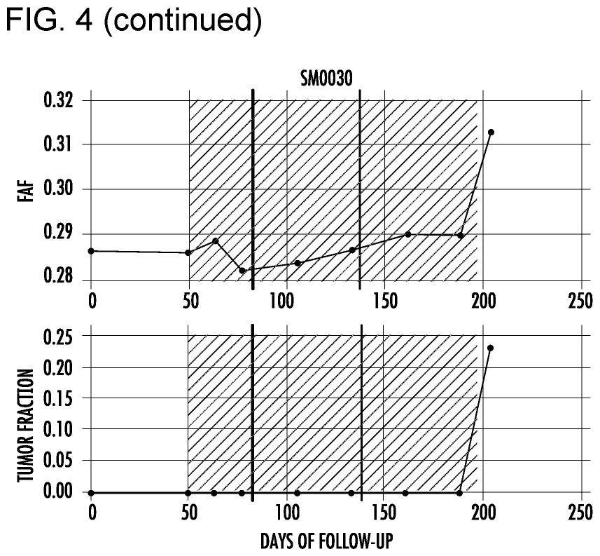

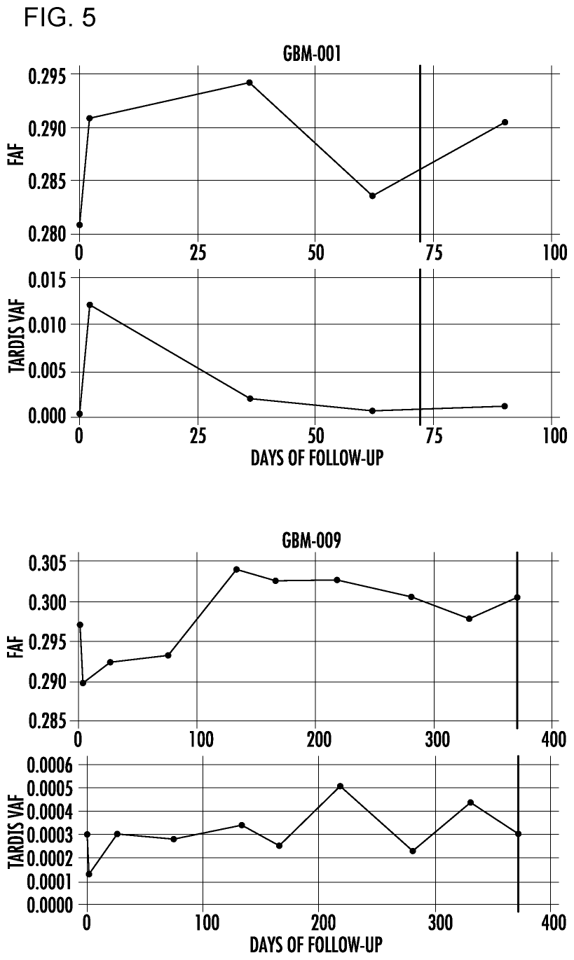

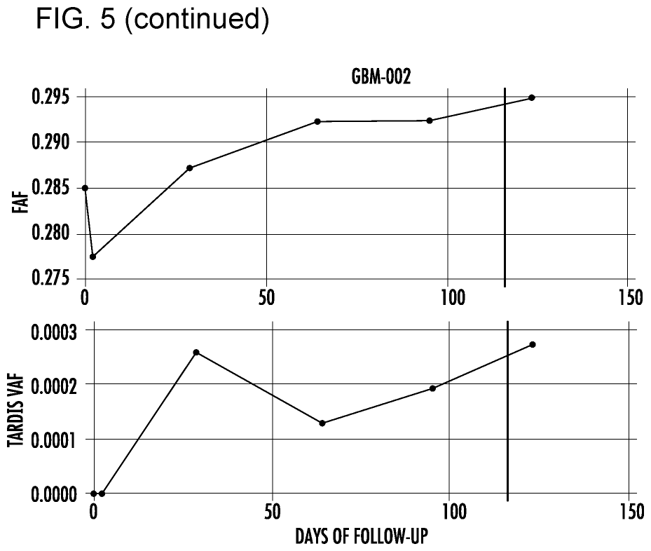

Compared to 40 plasma samples from healthy individuals, mean fraction of aberrant fragments (FAF) was higher in 261 samples from patients with melanoma (P=3.3×10⁻⁶), 46 samples from patients with cholangiocarcinoma (P=9.4×10⁻¹¹), 45 samples from patients with glioblastoma (P=9.3×10⁻⁹) and 47 samples from patients with breast cancer (P=4.5×10⁻⁸). To determine whether FAF was related to fraction of tumor DNA in plasma, we compared FAF with tumor fraction measured using analysis of copy number aberrations in patients with metastatic cancer. FAF was correlated with tumor fraction in patients with melanoma (r=0.76, P=9.9×10⁻⁵¹; FIG. 1B), and in patients with cholangiocarcinoma (r=0.71, P=2.2×10⁻⁸. FIG. 3). In FIG. 3, tumor fraction and FAF were correlated with Pearson's r of 0.71 (P=2.2×10−8). On the x-axis, plasma samples with tumor fraction below the limit of detection using ichorCNA are indicated as zero. In longitudinal samples from patients with metastatic melanoma, changes in FAF during therapy were consistent with changes in tumor fraction in plasma DNA (FIG. 1C, FIGS. 4A-4C and data S1). In some cases, changes in FAF were concordant with treatment response on imaging, even when tumor fraction in plasma DNA was undetectable using copy number analysis. In 3 patients with glioblastoma with multiple plasma samples available, we compared FAF with tumor fraction in plasma DNA measured using targeted digital sequencing(8). We found changes in FAF during therapy were consistent with changes in tumor fraction, even though detectable circulating tumor DNA concentrations were very low (0.01% to 1.2%; FIG. 1D, FIG. 5).

### Example 3. Confirmation of Tumors as Source of Aberrant cfDNA Fragments

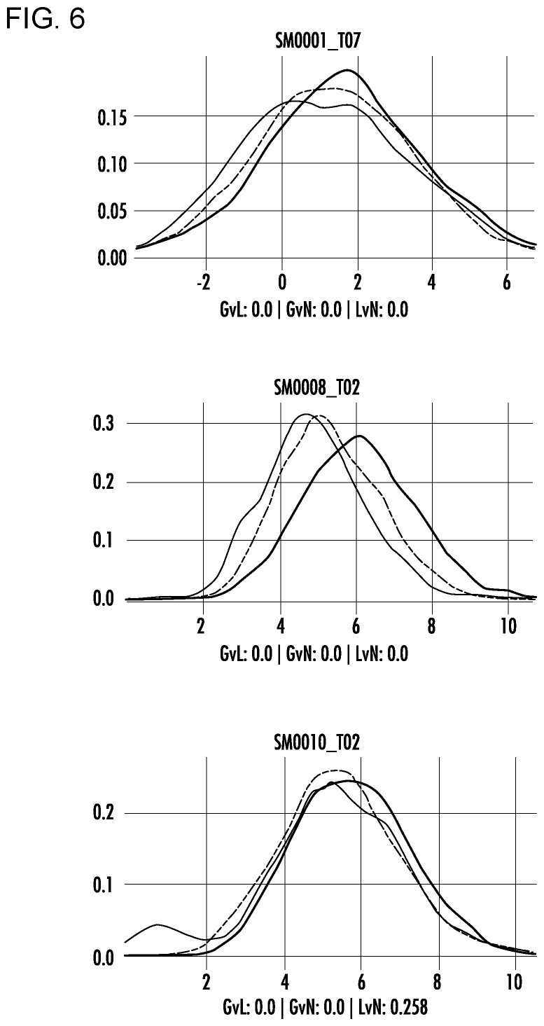

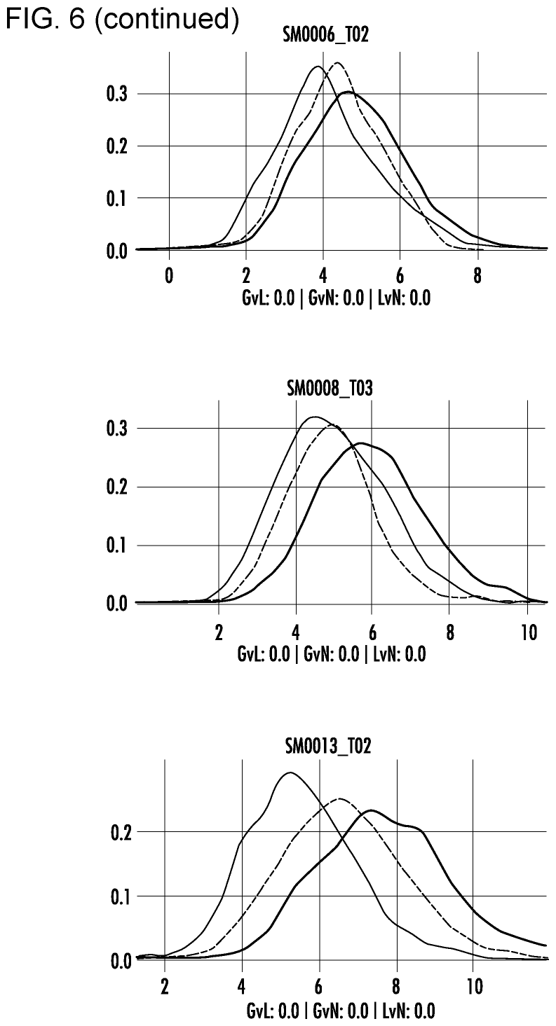

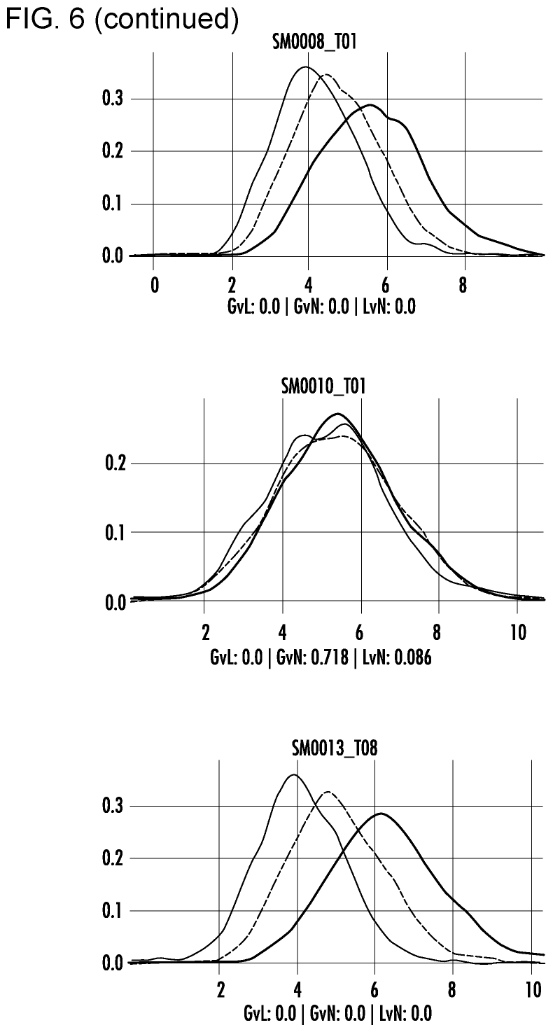

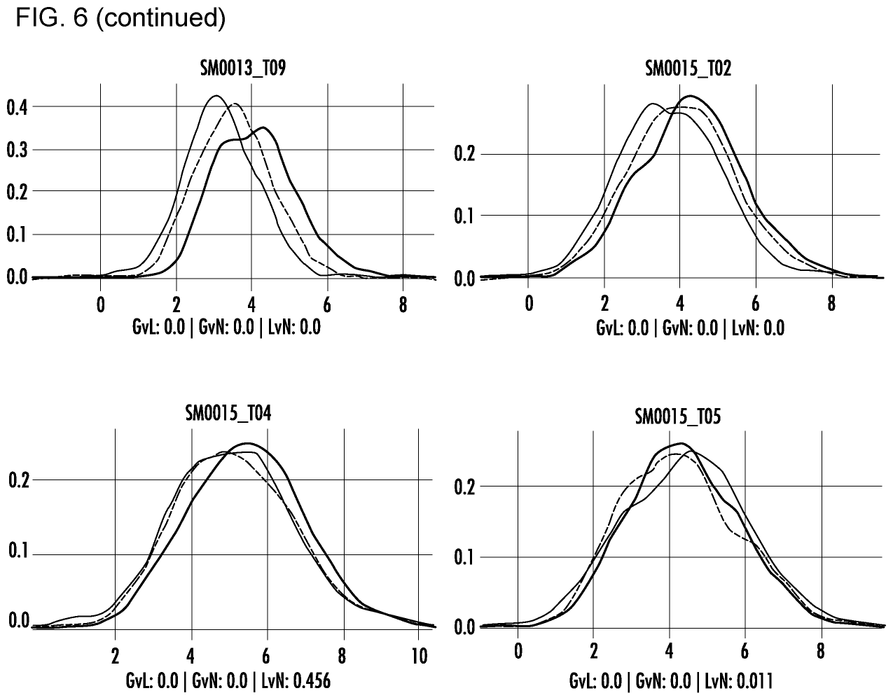

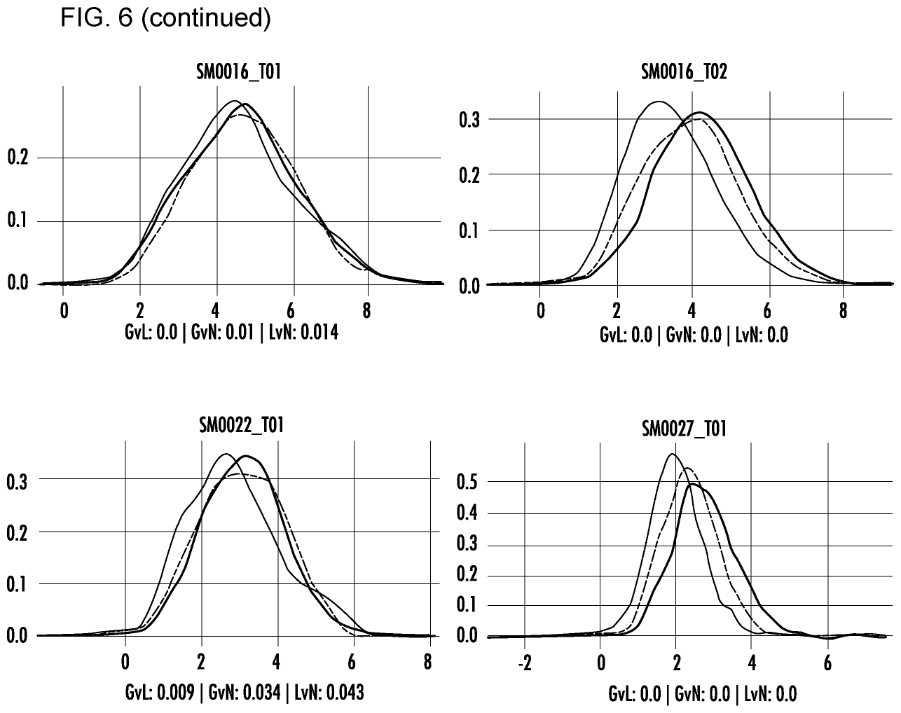

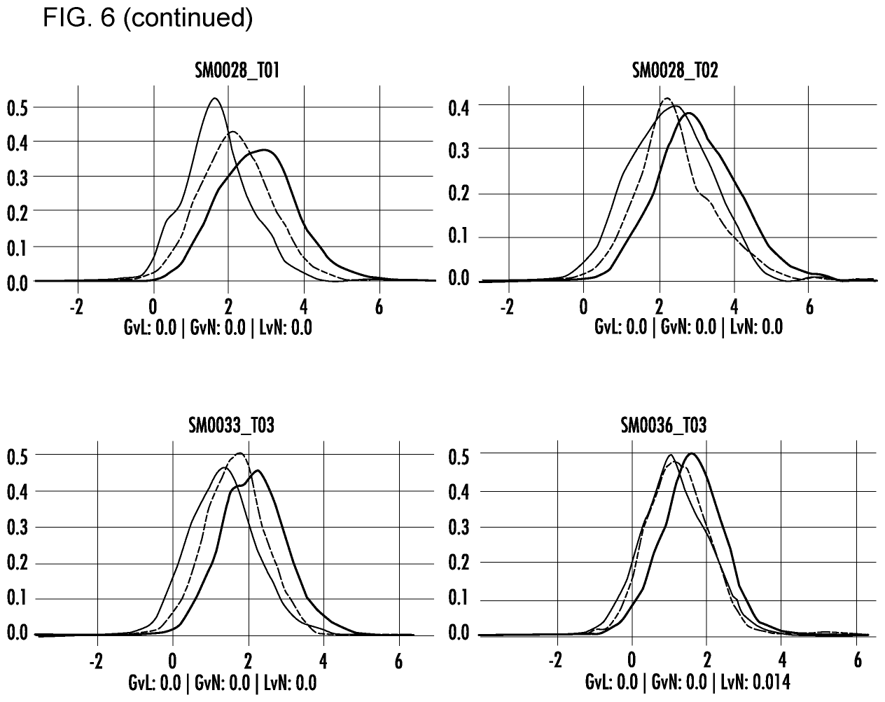

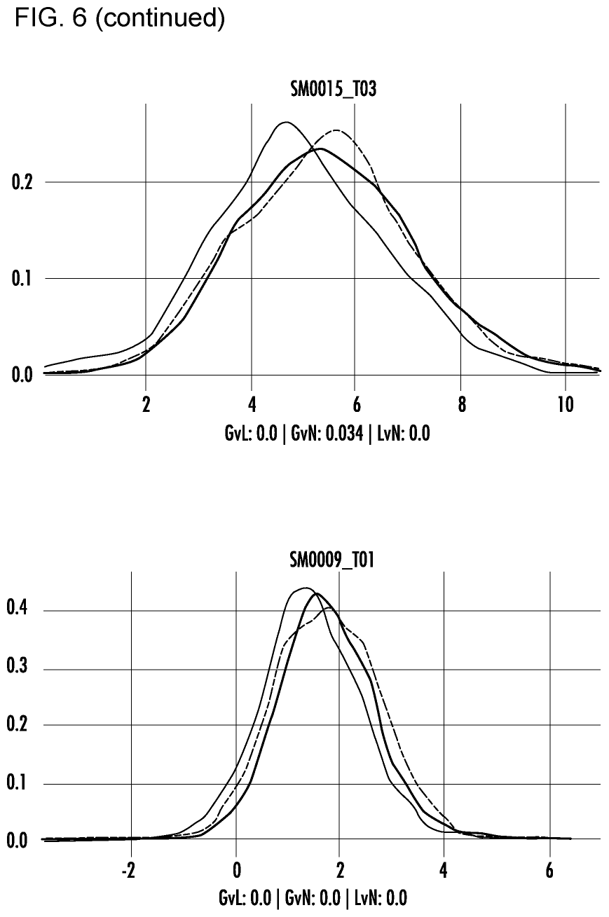

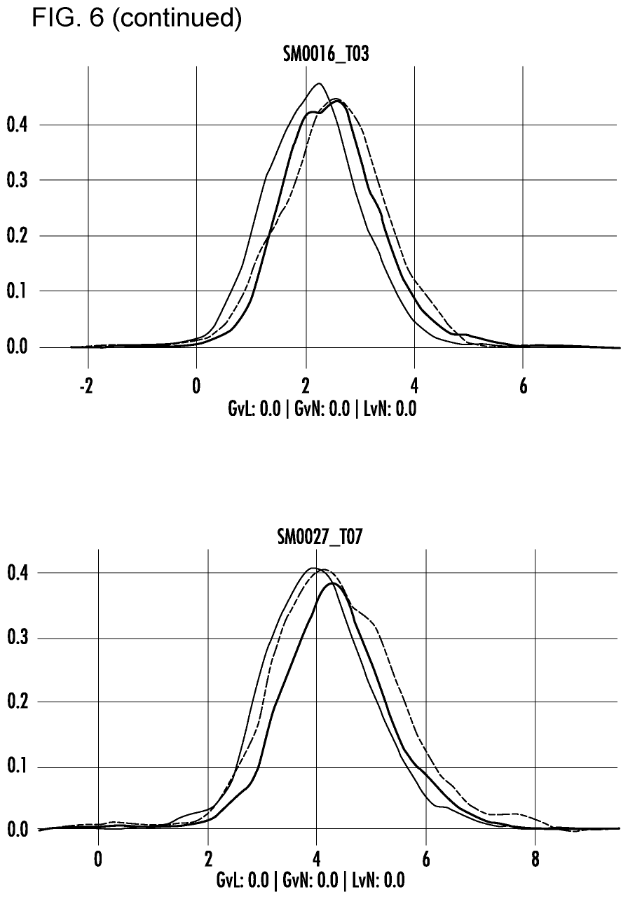

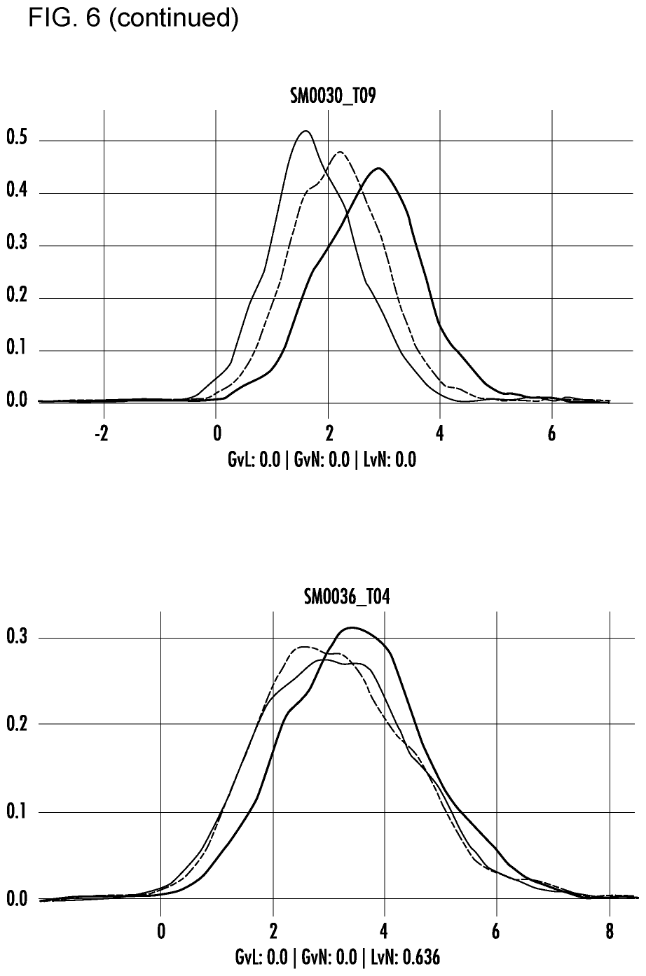

To ascertain that aberrant DNA fragments in plasma were contributed by the tumor, we focused our analysis on patients with metastatic melanoma and samples with high tumor fraction in plasma DNA. Since the tumor contributes more fragments of plasma DNA from genomic loci that are gained in the tumor genome compared to those that are lost, we expected FAF to be higher for genomic loci affected by copy number gains if tumor-derived DNA fragments in plasma are aberrant. In 27 plasma samples with at least 20% tumor fraction in plasma, we found that FAF was higher at genomic loci affected by copy number gains compared to loci unaffected by copy number changes or those affected by copy number losses (FIG. 1E and FIGS. 6A-6D). To further assess the tumor specificity of aberrant DNA fragments in plasma, we performed deep whole genome sequencing (>280×) for two plasma samples with tumor fractions of 36% and 39%. We evaluated whether DNA fragments carrying tumor-specific mutations were more likely to be aberrant (FIG. 1F and TABLE 2).

TABLE 2

| | | | Number of | FAF in | | | |
|---|---|---|---|---|---|---|---|
| | | | non- | non- | Number of | FAF in | |
| | Patient | Sample | mutated | mutated | mutated | mutated | |
| Diagnosis | ID | ID | fragments | fragments | fragments | fragments | P value |
| Melanoma | SM0008 | SM0008_T02 | 248405 | 0.334 | 79737 | 0.364 | <2.2 × |
| | | | | | | | 10⁻¹⁶ |
| Melanoma | SM0022 | SM0022_T01 | 297031 | 0.307 | 76809 | 0.328 | 1.5 × |
| | | | | | | | 10⁻¹¹ |

TABLE 2 shows a comparison of aberrant positioning between mutated and non-mutated fragments. Two-tailed p-values are reported from two proportions Z test.

In both plasma samples, we found that a greater fraction of mutated fragments were aberrant compared to non-mutated fragments covering the same genomic loci (P<2.2×10⁻¹⁶ and P=1.5×10⁻¹¹). Taken together, these results demonstrate that elevated fraction of aberrant fragments observed in plasma DNA from patients with cancer are driven by tumor-derived DNA fragments.

### Example 4. Evaluation of External Datasets to Validate Analytical Method

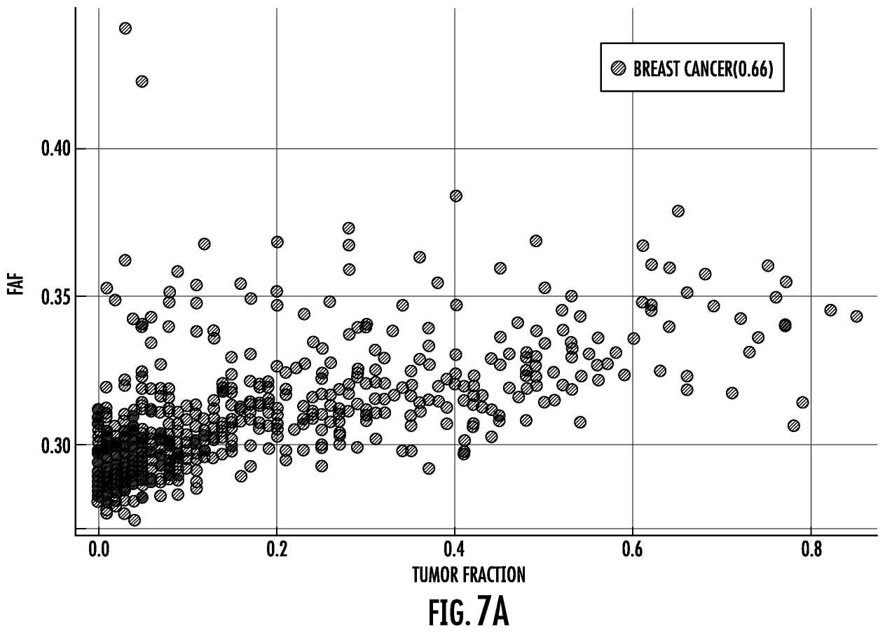

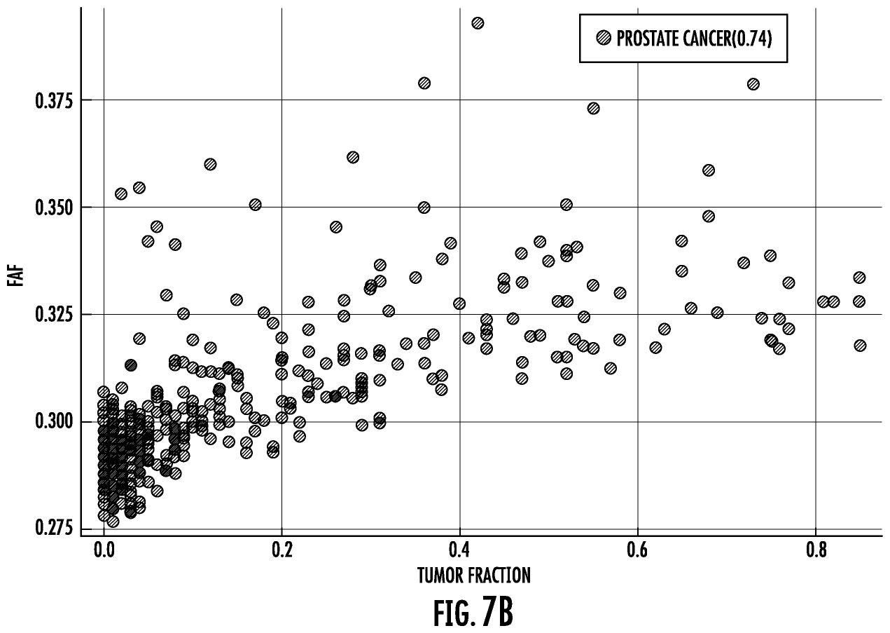

To evaluate external validity of our findings and ensure our observations were not driven by artifacts arising from sample handling and processing, we analyzed fragment ends in whole genome sequencing data from 3 recent publications, including 2270 plasma DNA samples from patients with cancer and healthy individuals (9-12). We found FAF was elevated across multiple cancer types and stages (FIG. 1A). In patients with metastatic breast and prostate cancer with high circulating tumor DNA concentrations, FAF was correlated with tumor fraction in plasma DNA (FIGS. 7A-7B). When compared to patients with liver cirrhosis and hepatitis, plasma samples from patients with liver cancer showed higher FAF (FIG. 1A). These results showed that analysis of fragment ends could be generalized across cancer types and remained relevant even for datasets that were generated independently from our laboratory.

### Example 5. Implementation of Machine Learning to Facilitate Genome-Wide Analysis of cfDNA Fragment Ends for Cancer Detection and Monitoring

To evaluate whether genome-wide analysis of fragment ends allows detection of cancer, we trained a machine learning model based on random forests to classify plasma samples from cancer patients and healthy individuals. Using our approach for calculation of FAF, whether ends of a fragment are aberrant can only be determined if the fragment maps to a genomic locus known to be protected from degradation in healthy individuals. This approach excludes any fragments that map to other unannotated regions of the genome, limiting the proportion of informative data to a mean of 34% from plasma DNA samples. Differences in genomic positioning of plasma DNA fragments can also be captured through analysis of nucleotide sequence at fragment ends.

To utilize all mapped plasma DNA fragments in our classification model, we included nucleotide frequencies observed 10 bp upstream and downstream of fragment ends (based on the reference genome sequence), averaged across all fragments for each sample. To assess whether changes in average nucleotide frequencies at fragment ends were driven by tumor contribution in plasma DNA, we used multidimensional scaling and compared the first two dimensions of nucleotide frequencies with FAF and with tumor fraction for 4 cohorts of patients with advanced cancers and high circulating tumor DNA concentrations. Absolute values for correlation between the second dimension of nucleotide frequencies at fragment ends and FAF were 0.70, 0.73, 0.71 and 0.48 for patients with melanoma, cholangiocarcinoma, breast cancer and prostate cancer, respectively. In similar analysis, correlation between nucleotide frequencies at fragment ends and tumor fraction in plasma was 0.55, 0.55, 0.57 and 0.45, respectively (see TABLE 3), suggesting that changes in average nucleotide frequencies at fragment ends were, at least in part, driven by tumor contribution in plasma DNA.

TABLE 3 shows a correlation of nucleotide frequencies at fragment ends with tumor fraction and FAF in plasma DNA. Correlation between dimension 2 of nucleotide frequencies at fragment ends with tumor fraction and with FAF were all statistically significant (P<0.05).

TABLE 3

| | | | Nucleotide frequencies | |
|---|---|---|---|---|
| | | | at fragment ends | |
| | | | Dimen- | Dimen- |
| | | | sion 1 | sion 2 |
| Correlation | This study | CCA | −0.169 | 0.551 |
| with tumor | | Melanoma | 0.085 | 0.551 |
| fraction | Adalsteinsson | Breast Cancer | 0.002 | −0.571 |
| | et al. | Prostate Cancer | 0.099 | −0.447 |
| Correlation | This study | CCA | 0.108 | 0.735 |
| with FAF | | Melanoma | 0.183 | 0.700 |
| | Adalsteinsson | Breast Cancer | 0.004 | −0.712 |
| | et al. | Prostate Cancer | 0.106 | −0.476 |

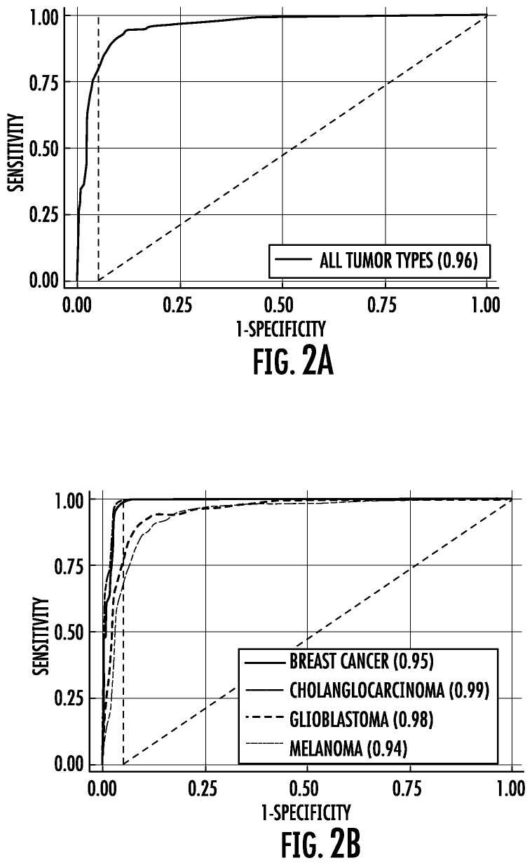

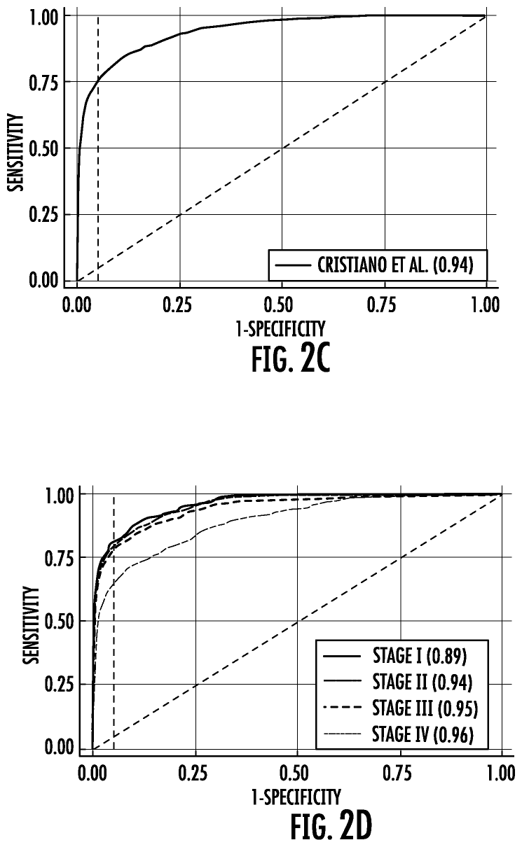

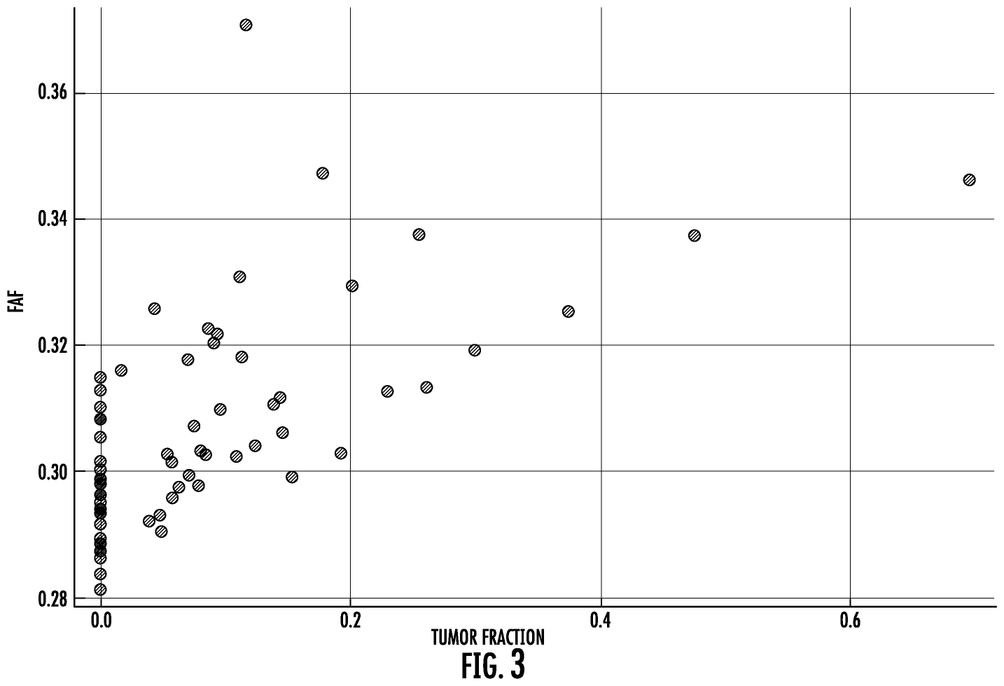

To avoid overfitting our classification model, we restricted the analysis to the earliest available plasma sample for each patient (generally obtained at enrollment in the clinical study). This analysis was averaged over 100 runs, using 80% of samples for training and 20% for testing in each iteration (split proportionally for each cancer type and cohort). Across all patients in our cohort, analysis of fragment ends achieved an area under the receiver operating characteristic curve (AUC) value of 0.96 (FIG. 2A). Performance varied across cancer types and AUC values for melanoma, cholangiocarcinoma, breast cancer, and glioblastoma were 0.94, 0.99, 0.95, and 0.98 respectively (FIG. 2B). At 95% specificity, sensitivity for cancer detection was 79% for all cancer types, 68% for melanoma, 78% for breast cancer, 99% for glioblastoma and 99% for cholangiocarcinoma.

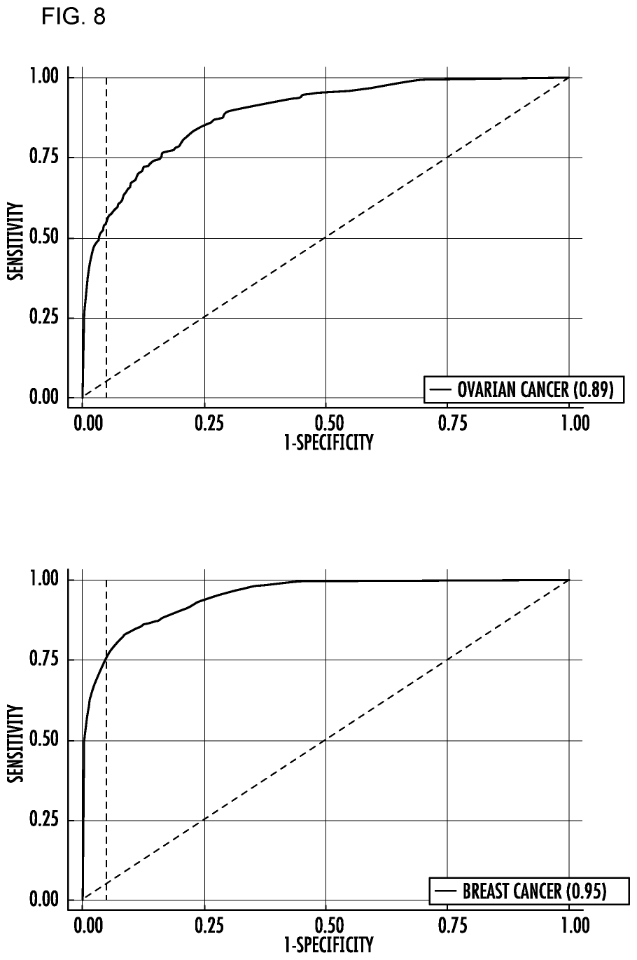

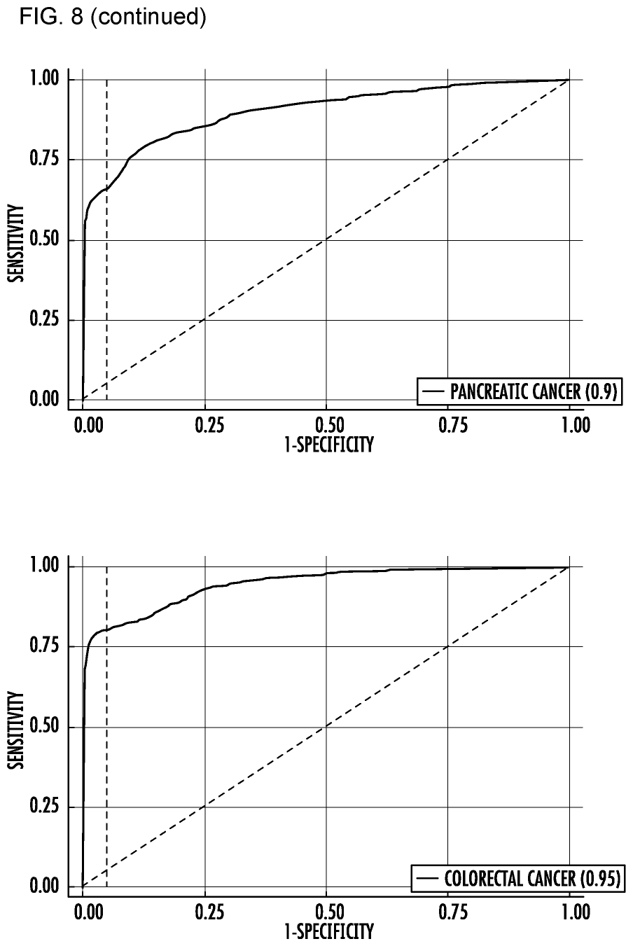

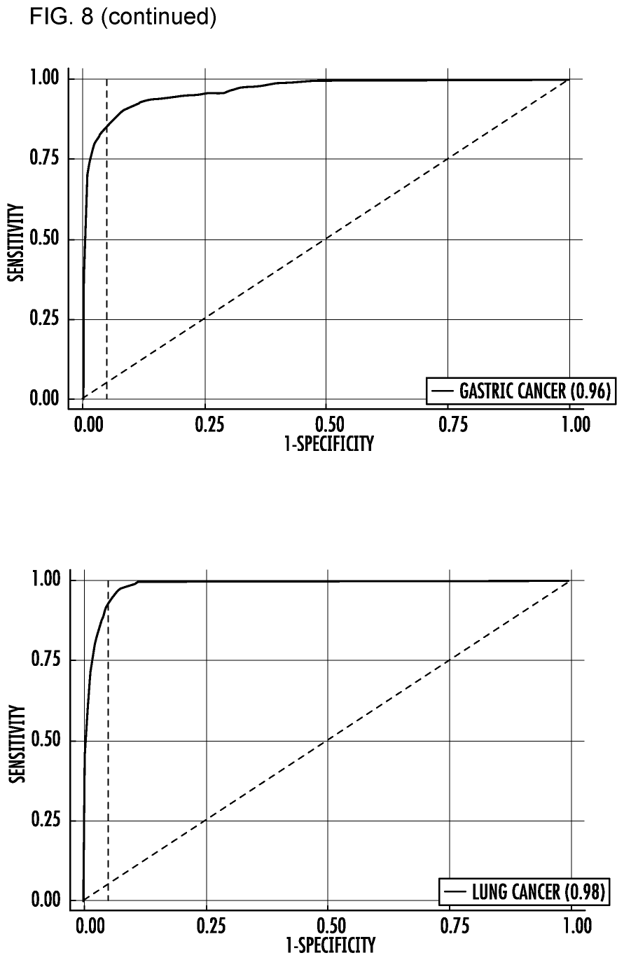

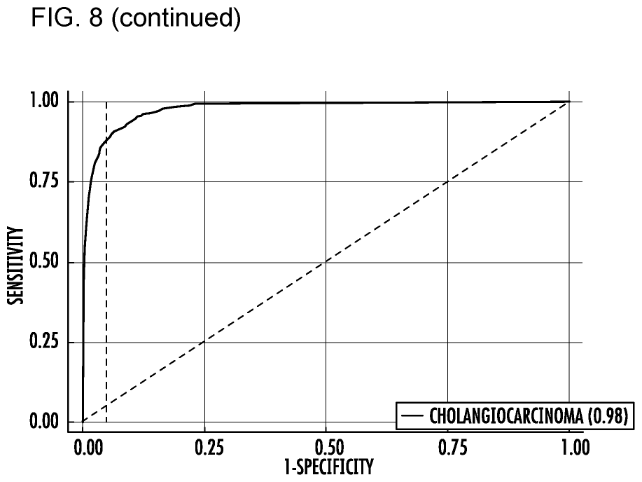

To evaluate whether analysis of fragment ends was generalizable for cancer detection beyond our own samples, we implemented this approach using plasma whole genome sequencing data from a recent publication (12) that included a greater number of healthy individuals, multiple cancer types and more samples from patients with stage I-III cancers. Following a similar setup for training and testing, we found an AUC value of 0.94 across all patients (FIG. 2C). At 95% specificity, sensitivity for cancer detection was 76% for all cancer types, 59% for ovarian cancer, 67% for pancreatic cancer, 75% for breast cancer, 80% for colorectal cancer, 86% for gastric cancer, 90% for cholangiocarcinoma and 97% for lung cancer (FIG. 8A-8B). When this analysis was limited to patients with potentially curable Stage I-III disease, AUC value dropped marginally to 0.93, with 75% sensitivity at 95% specificity (FIG. 2D).

### Example 6. Down-Sampling of Sequencing Data to Determine Cost-Effectiveness of Analysis for Cancer Detection and Monitoring

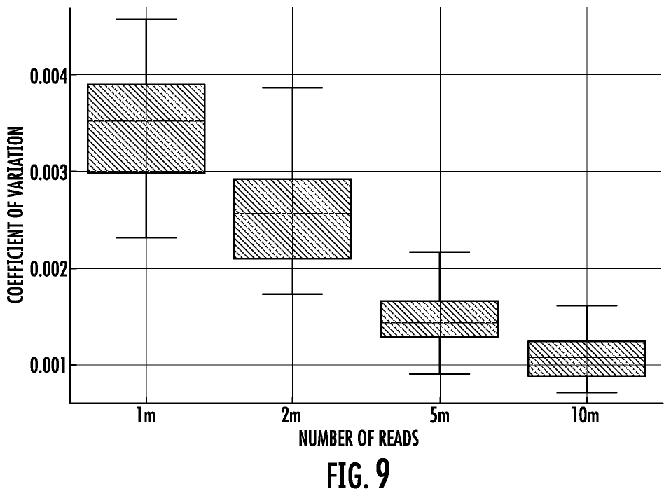

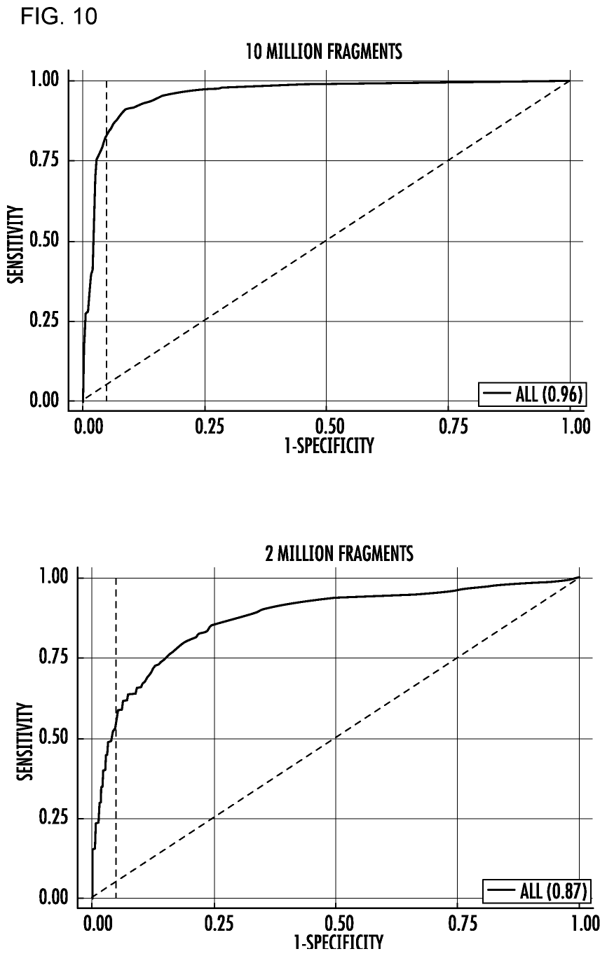

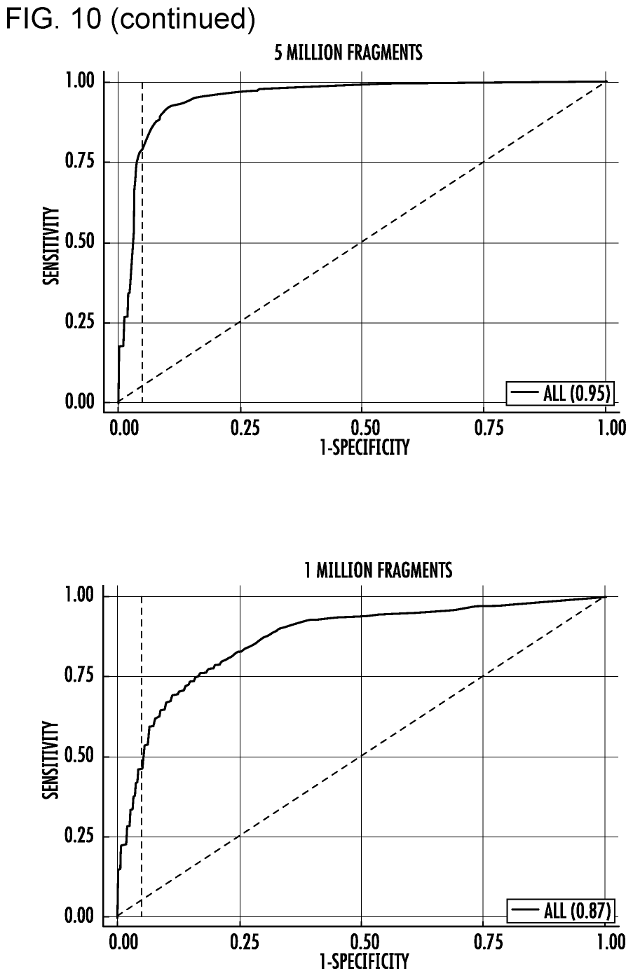

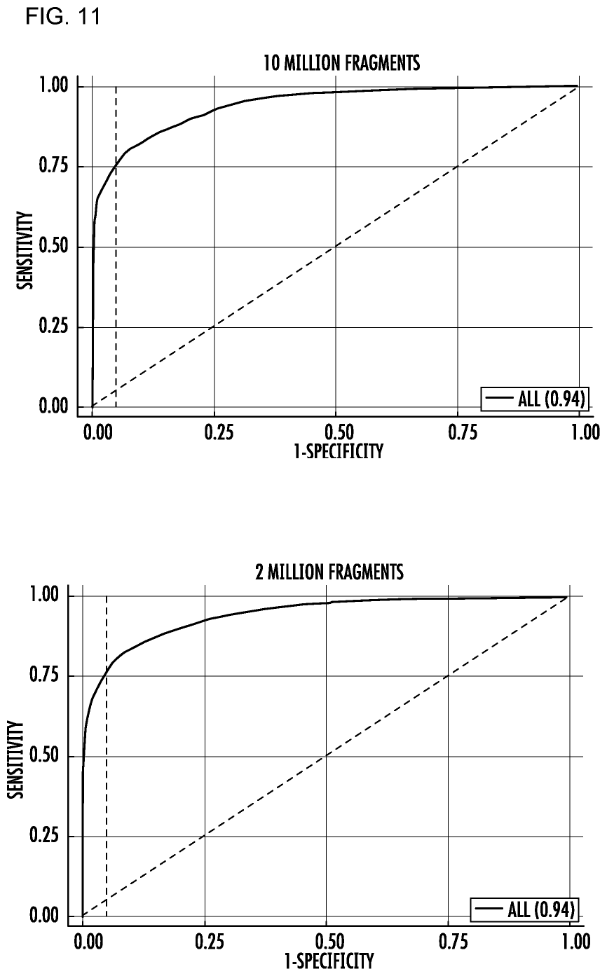

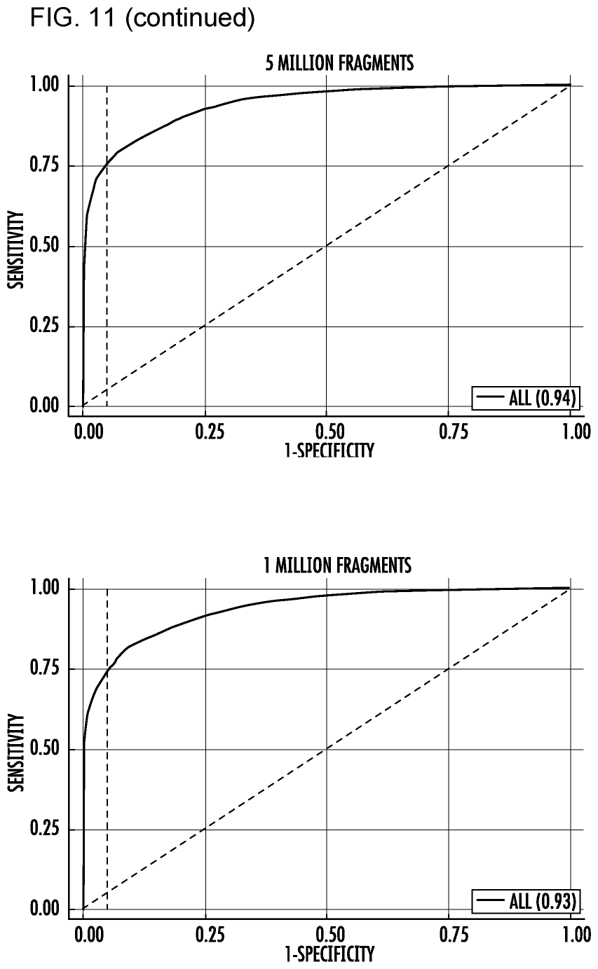

To determine whether analysis of fragment ends in plasma DNA would be cost-effective as a biomarker, we down-sampled sequencing data, and evaluated measurement of FAF and performance of the classifier at multiple read depths. Even when only a maximum of 1 million fragments were analyzed, we found that co-efficient of variation for FAF was less than 1% in independent sets of fragments from the same plasma samples (FIG. 9). When fragments per sample were limited to 1 million, AUC values obtained for the random forests classifier were 0.87 for our multi-cancer cohort and 0.93 for published data(12), respectively (FIG. 10 and FIG. 11).

### Example 7. Summary and Further Analysis of Experimental Results

Overall, these results demonstrate that ends of tumor-derived plasma DNA fragments are more likely to be observed at different genomic loci compared to ends of background DNA fragments contributed by peripheral blood cells. We leveraged this observation and showed proof-of-principle results that analysis of fragment ends can be useful as a biomarker for cancer detection and monitoring of treatment response. This approach appears potentially useful for several cancer types where detection of cancer at earlier stages could improve outcomes where there are no established methods for screening, including cholangiocarcinoma, pancreatic cancer, gastric cancer and ovarian cancer. In addition, the diagnostic performance of this approach in patients with glioblastoma is particularly surprising, given how challenging circulating tumor DNA detection has been for these patients using mutation-based assays. A potential explanation for this finding is that analysis of fragment ends leverages differences in cell-free DNA shedding from different tissue types in healthy individuals and patients with cancer. Hence, this approach may perform better for cancers originating in tissues that rarely contribute cell-free DNA into plasma within healthy individuals. However, this also suggests a potential limitation that aberrant fragmentation patterns in plasma may not be specific to cancer and may arise from unexpected tissue contributions in plasma due to other systemic or acute conditions including pregnancy and transplant (13). In our analysis, we did not find elevated FAF in plasma samples from patients with liver cirrhosis or hepatitis, and a higher FAF was observed in patients with hepatocellular carcinoma.

To utilize this approach for cancer detection, a reference dataset may be needed that includes healthy individuals across age, gender and co-morbidities. In addition, each patient's results may need to be obtained when they are unaffected by acute illness and interpreted in the appropriate clinical context. Our approach can be improved further through analysis of even larger number of samples from patients across disease stages for each cancer type to increase accuracy of cancer detection. In the future, such data may also be useful to predict tumor type for plasma samples from cancer patients, either through selection of the most informative genomic regions to calculate FAF, and by identifying cancer type-specific nucleotide motifs and frequencies at fragment ends.

Earlier studies of fragmentation patterns in circulating tumor DNA have evaluated local differences in average fragment size in windows across the genome as an independent approach for cancer detection (12), or used fragment size to improve sensitivity for detection of somatic genomic alterations (14, 15). Another study identified genomic loci (16) and nucleotide motifs (13) preferentially utilized by DNA shed from liver cells and found liver-derived DNA was higher in patients with hepatocellular carcinoma. In contrast to these studies, we have found that fraction of aberrant fragments and average nucleotide frequencies at fragment ends, measured in aggregate for each sample, can serve as a biomarker for multiple cancer types. Analyzing just one million fragments per sample from plasma whole genome sequencing libraries, we have found that the performance of our approach for cancer detection parallels published methods based on more complex analysis of mutations (17), methylation (18) and fragment size (12) that require higher amounts of input DNA and greater depth of sequencing.

The simplicity of our approach as well as the small amount of plasma DNA and sequencing data required can greatly increase access to blood-based cancer detection and monitoring, particularly for resource-constrained health systems. Our results serve as a valuable proof-of-principle. Prospective clinical studies evaluating performance of analysis of fragment ends for early detection and for monitoring treatment response in patients with cancer will further confirm and validate these results.

### Example 8. Reduction of the Number of Features Input into the Machine Learning

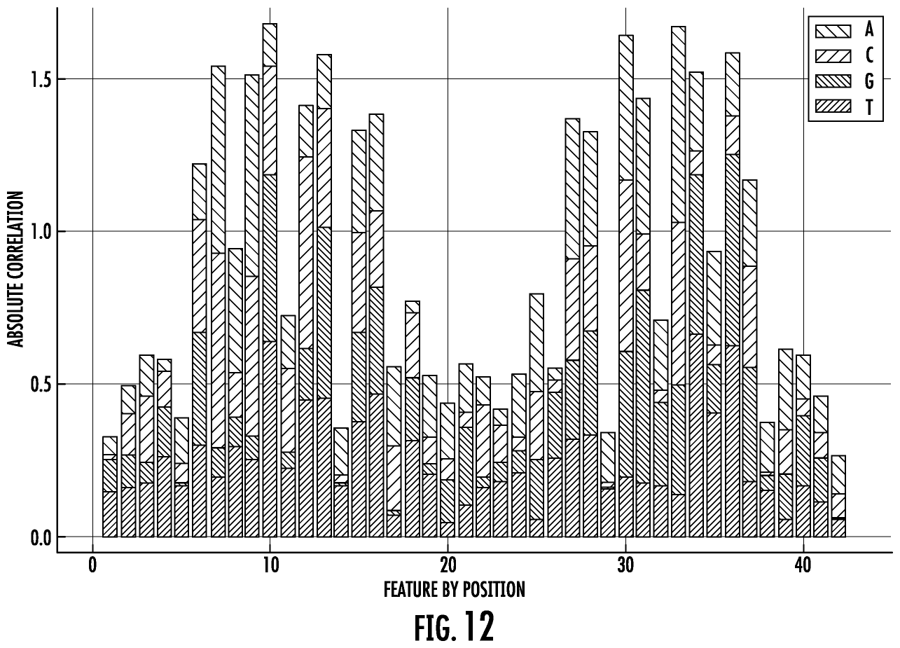

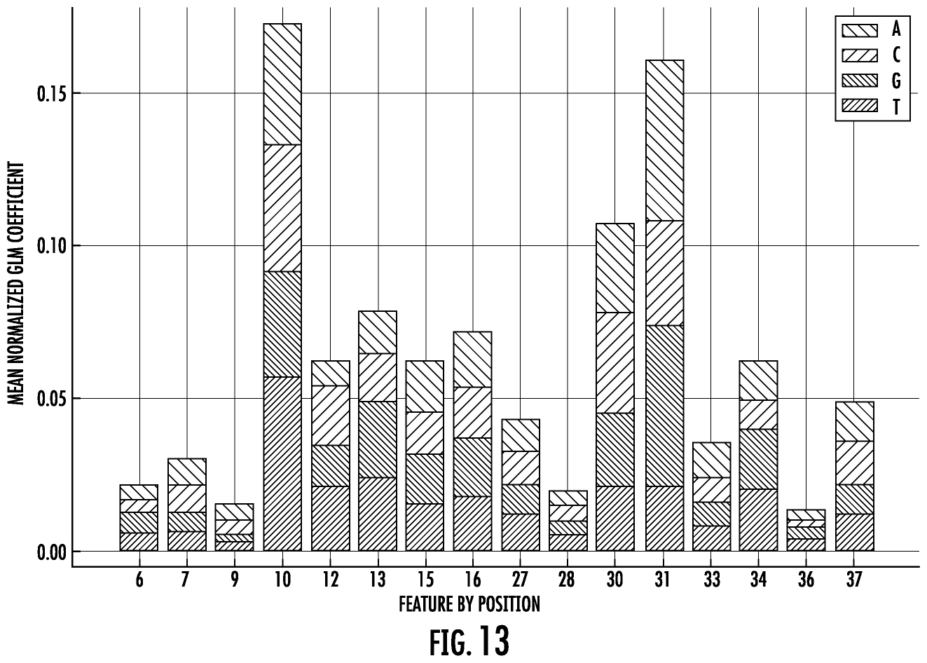

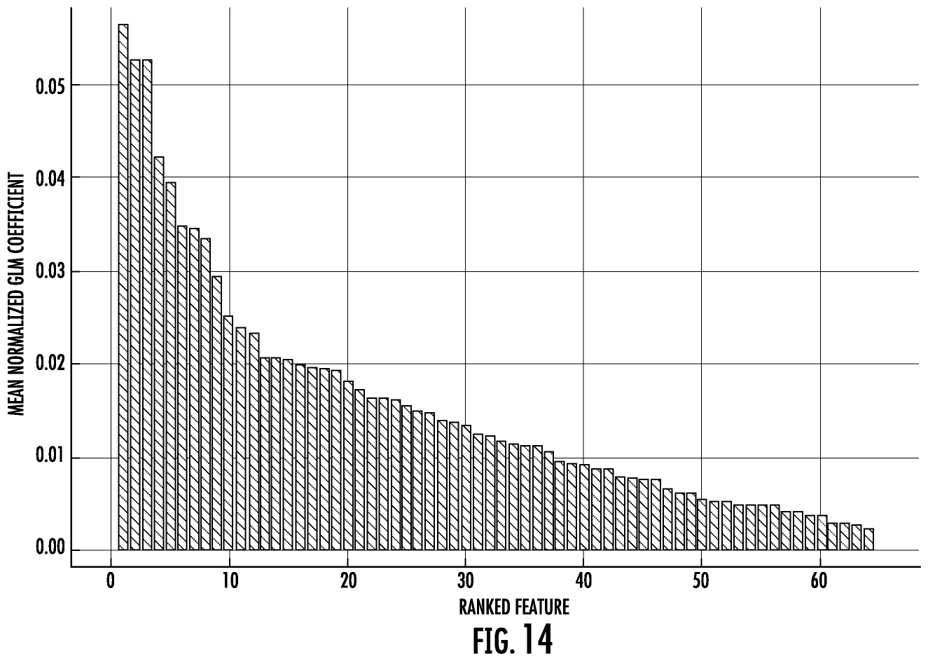

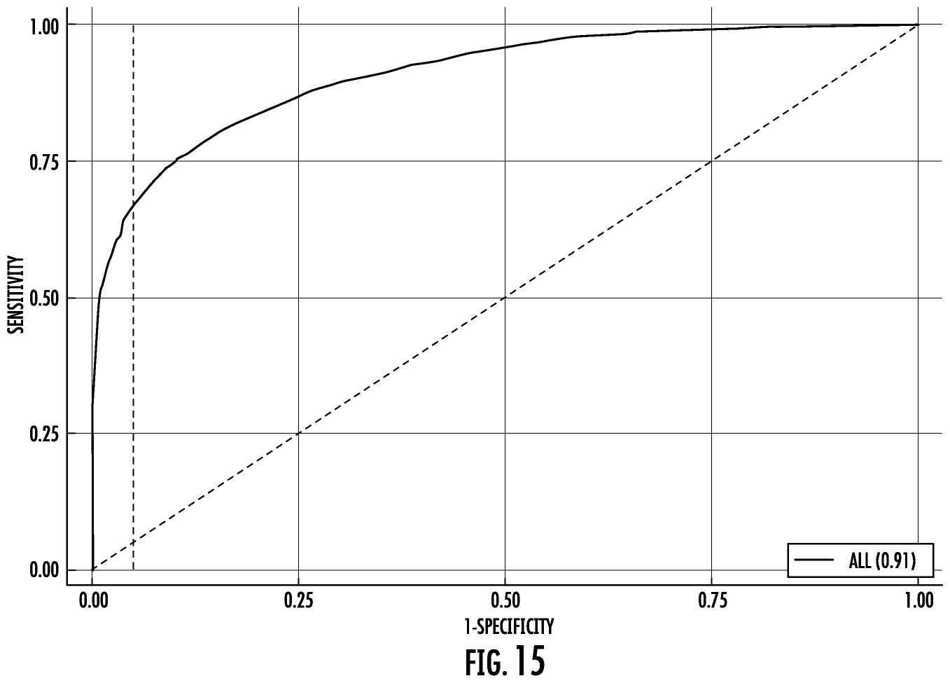

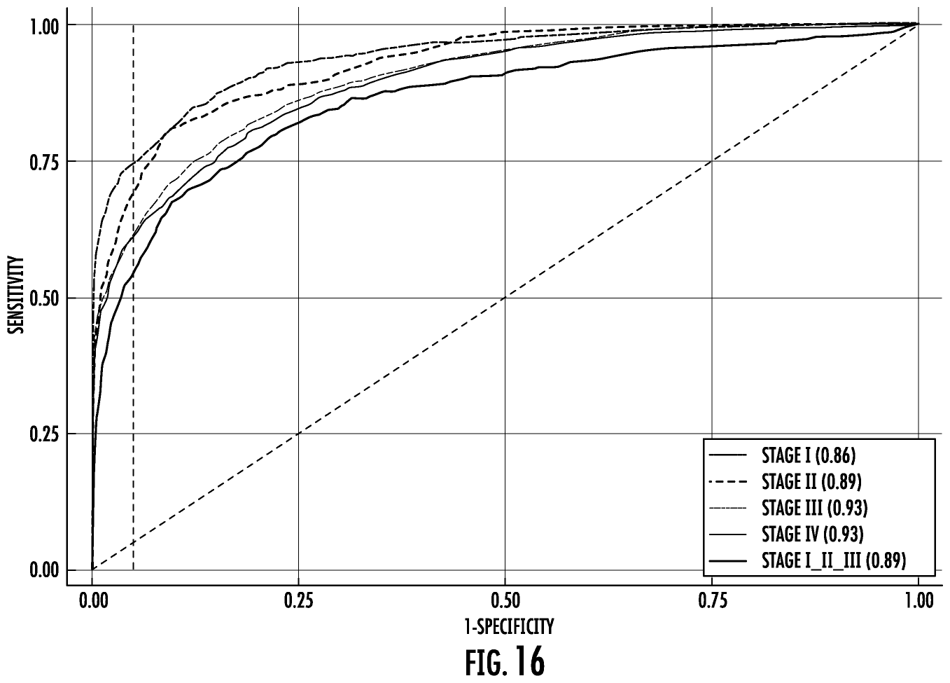

An experiment was conducted to evaluate selection of specific nucleotide frequencies (rather than all 168 features) to feed into the machine learning model. It was observed that this selection can be made by determining which nucleotide frequencies are most correlated with tumor fraction and FAF in a subset of samples (see FIGS. 12-13). This experiment demonstrated the ability to reduce the number of features that go into the machine learning from 168 to just 9 of the most important (from just 3 positions around the fragment ends) (see FIGS. 14-16). This represents a significant improvement in the nucleotide frequency approach.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### REFERENCES

1. F. C. Wong, Y. M. Lo, Prenatal Diagnosis Innovation: Genome Sequencing of Maternal Plasma. Annu Rev Med 67, 419-432 (2016).
2. P. Burnham, K. Khush, I. De Vlaminck, Myriad Applications of Circulating Cell-Free DNA in Precision Organ Transplant Monitoring. Ann Am Thorac Soc 14, S237-S241 (2017).
3. Y. van der Pol, F. Mouliere, Toward the Early Detection of Cancer by Decoding the Epigenetic and Environmental Fingerprints of Cell-Free DNA. Cancer Cell 36, 350-368 (2019).
4. M. W. Snyder, M. Kircher, A. J. Hill, R. M. Daza, J. Shendure, Cell-free DNA Comprises an In Vivo Nucleosome Footprint that Informs Its Tissues-Of-Origin. Cell 164, 57-68 (2016).
5. K. Sun et al., Plasma DNA tissue mapping by genome-wide methylation sequencing for noninvasive prenatal, cancer, and transplantation assessments. Proc Natl Acad Sci USA 112, E5503-5512 (2015).
6. H. Markus et al., Analysis of recurrently protected genomic regions in cell-free DNA found in urine. Sci Transl Med 13, (2021).
7. D. A. Cusanovich et al., A Single-Cell Atlas of In Vivo Mammalian Chromatin Accessibility. Cell 174, 1309-1324 e1318 (2018).
8. B. R. McDonald et al., Personalized circulating tumor DNA analysis to detect residual disease after neoadjuvant therapy in breast cancer. Sci Transl Med 11, (2019).
9. H. Zheng, M. S. Zhu, Y. Liu, FinaleDB: a browser and database of cell-free DNA fragmentation patterns. Bioinformatics, (2020).
10. V. A. Adalsteinsson et al., Scalable whole-exome sequencing of cell-free DNA reveals high concordance with metastatic tumors. Nat Commun 8, 1324 (2017).
11. P. Jiang et al., Lengthening and shortening of plasma DNA in hepatocellular carcinoma patients. Proc Natl Acad Sci USA 112, E1317-1325 (2015).
12. S. Cristiano et al., Genome-wide cell-free DNA fragmentation in patients with cancer. Nature 570, 385-389 (2019).
13. P. Jiang et al., Plasma DNA End-Motif Profiling as a Fragmentomic Marker in Cancer, Pregnancy, and Transplantation. Cancer Discov 10, 664-673 (2020).
14. F. Mouliere et al., Enhanced detection of circulating tumor DNA by fragment size analysis. Sci Transl Med 10, (2018).
15. A. Zviran et al., Genome-wide cell-free DNA mutational integration enables ultra-sensitive cancer monitoring. Nat Med 26, 1114-1124 (2020).
16. P. Jiang et al., Preferred end coordinates and somatic variants as signatures of circulating tumor DNA associated with hepatocellular carcinoma. Proc Natl Acad Sci USA 115, E10925-E10933 (2018).
17. J. D. Cohen et al., Detection and localization of surgically resectable cancers with a multi-analyte blood test. Science 359, 926-930 (2018).
18. M. C. Liu et al., Sensitive and specific multi-cancer detection and localization using methylation signatures in cell-free DNA. Ann Oncol 31, 745-759 (2020).
19. P. M. LoRusso et al., Identifying treatment options for BRAFV600 wild-type metastatic melanoma: A SU2C/MRA genomics-enabled clinical trial. PLOS One 16, e0248097 (2021).
20. S. A. Byron et al., Prospective Feasibility Trial for Genomics-Informed Treatment in Recurrent and Progressive Glioblastoma. Clin Cancer Res 24, 295-305 (2018).
21. H. Markus et al., Evaluation of pre-analytical factors affecting plasma DNA analysis. Sci Rep 8, 7375 (2018).
22. S. Chen, Y. Zhou, Y. Chen, J. Gu, fastp: an ultra-fast all-in-one FASTQ preprocessor. Bioinformatics 34, 1884-1890 (2018).
23. H. Li, Aligning sequence reads, clone sequences and assembly contigs with BWA-MEM. arXiv preprint arXiv: 1303.3997, (2013).
24. H. Li et al., The Sequence Alignment/Map format and SAMtools. Bioinformatics 25, 2078-2079 (2009).
25. V. A. Adalsteinsson et al., Scalable whole-exome sequencing of cell-free DNA reveals high concordance with metastatic tumors. Nat Commun 8, 1324 (2017).
26. G. Ha et al., Integrative analysis of genome-wide loss of heterozygosity and monoallelic expression at nucleotide resolution reveals disrupted pathways in triple-negative breast cancer. Genome Res 22, 1995-2007 (2012).
27. S. Cristiano et al., Genome-wide cell-free DNA fragmentation in patients with cancer. Nature 570, 385-389 (2019).
28. P. Jiang et al., Lengthening and shortening of plasma DNA in hepatocellular carcinoma patients. Proc Natl Acad Sci USA 112, E1317-1325 (2015).
29. H. Zheng, M. S. Zhu, Y. Liu, FinaleDB: a browser and database of cell-free DNA fragmentation patterns. Bioinformatics, (2020).
30. M. W. Snyder, M. Kircher, A. J. Hill, R. M. Daza, J. Shendure, Cell-free DNA Comprises an In Vivo Nucleosome Footprint that Informs Its Tissues-Of-Origin. Cell 164, 57-68 (2016).
31. A. R. Quinlan, I. M. Hall, BEDTools: a flexible suite of utilities for comparing genomic features. Bioinformatics 26, 841-842 (2010).
32. S. Heinz et al., Simple combinations of lineage-determining transcription factors prime cis-regulatory elements required for macrophage and B cell identities. Mol Cell 38, 576-589 (2010).
33. N. Wan et al., Machine learning enables detection of early-stage colorectal cancer by whole-genome sequencing of plasma cell-free DNA. BMC Cancer 19, 832 (2019).
34. B. R. McDonald et al., Personalized circulating tumor DNA analysis to detect residual disease after neoadjuvant therapy in breast cancer. Sci Transl Med 11, (2019).

What is claimed is:

1. A method of detecting disease in a patient, the method comprising the steps of:
obtaining a sample from the patient;
extracting cell-free DNA (cfDNA) from the sample to obtain cfDNA fragments;
performing sequencing on the cfDNA fragments extracted from the sample to generate sequencing reads for the cfDNA fragments;
determining an average nucleotide frequency at start sites and end sites of the cfDNA fragments;
determining a fraction of aberrant fragments in the cfDNA fragments from the sample;
inputting the average nucleotide frequency and the fraction of aberrant fragments into a machine learning classifier trained using genomic data from both healthy and diseased subjects; and
determining presence of the disease in the patient based on output of the machine learning classifier.

2. The method of claim 1, further comprising generating the machine learning classifier by training the machine learning classifier using fractions of aberrant fragments in cfDNA from healthy subjects and using fractions of aberrant fragments in cfDNA from diseased subjects.

3. The method of claim 2, further comprising training the machine learning classifier using average nucleotide frequency at start sites and end sites in cfDNA from healthy subjects and using average nucleotide frequency at start sites and end sites in cfDNA from diseased subjects.

4. The method of any of claims 1-3, wherein the machine learning classifier is trained using genomic data from the earliest available samples from healthy and diseased subjects.

5. The method of any of claims 1-4, wherein the machine learning classifier is trained using genomic data comprising a reference dataset from healthy subjects across age, gender and co-morbidities corresponding with those of the diseased subjects.

6. The method of any of claims 1-5, wherein the machine learning classifier is trained using genomic data comprising a dataset from diseased subjects across disease stages and/or disease types.

7. The method of any of claims 1-6, wherein analysis of as few as one million fragments per sample, as few as 900,000 fragments per sample, as few as 800,000 fragments per sample, as few as 700,000 fragments per sample, as few as 600,000 fragments per sample, or as few as 500,000 fragments per sample from whole genome sequencing libraries allows for detection of the disease.

8. The method of any one of claims 1-7, wherein the disease is cancer.

9. The method of claim 8, wherein the cancer is a cancer with no established methods for screening selected from the group consisting of cholangiocarcinoma, pancreatic cancer, gastric cancer, and ovarian cancer.

10. The method of claim 8, wherein the cancer is selected from the group consisting of melanoma, cholangiocarcinoma, glioblastoma, breast cancer, prostate cancer, colorectal cancer, gastric cancer, lung cancer, and ovarian cancer.

11. The method of any of claims 1-10, wherein the sample is plasma, urine, or cerebrospinal fluid.

12. The method of any of claims 1-11, wherein the patient is human.

13. The method of any of claims 1-11, wherein the patient is a dog or a cat.

14. The method of claims 1-11, wherein the healthy and diseased subjects are non-human.

15. The method of claim 14, wherein the healthy and diseased subjects include dogs or cats.

16. The method of any of claims 1-15, wherein the machine learning classifier comprises a random forest, a support vector machine (SVM), a boosting algorithm, a gradient boost method (GBM), an extreme gradient boost method (XGBoost)), and/or a neural network.

17. The method of claim 16, wherein the machine learning classifier comprises a random forest.

18. The method of claim 16 or 17, wherein the machine learning classifier comprises a gradient boosted tree and/or a neural network.

19. The method of any of claims 1-18, wherein the method is computer-implemented.

20. A method of detecting disease in a patient, the method comprising the steps of:
obtaining a sample from the patient;
extracting cell-free DNA (cfDNA) from the sample to obtain cfDNA fragments;
performing sequencing on the cfDNA fragments extracted from the sample to generate sequencing reads for the cfDNA fragments;
determining a nucleotide frequency at start sites and end sites of the cfDNA fragments;
generating a nucleotide frequency vector from the nucleotide frequency at start sites and end sites;
determining a fraction of aberrant fragments in the cfDNA fragments from the sample;
inputting the nucleotide frequency vector and the fraction of aberrant fragments into a random forest classifier trained using genomic data from both healthy and diseased subjects; and
determining presence of the disease in the patient based on output of the random forest classifier.

21. The method of claim 20, further comprising generating the random forest classifier by training the random forest classifier using fractions of aberrant fragments in cfDNA from healthy subjects and using fractions of aberrant fragments in cfDNA from diseased subjects.

22. The method of claim 20 or 21, further comprising training the random forest classifier using a vector of nucleotide frequency at start sites and end sites in cfDNA from healthy subjects and using a vector of nucleotide frequency at start sites and end sites in cfDNA from diseased subjects.

23. The method of any of claims 20-22, further comprising training the random forest classifier using a nucleotide frequency at start sites and end sites in cfDNA from a sample taken from the subject at an earlier point in time.

24. The method of any of claims 20-23, further comprising training the random forest classifier using a fraction of aberrant fragments in cfDNA from the sample taken from the subject at the earlier point in time.

25. The method of any of claims 20-24, wherein the disease is cancer.

26. The method of any of claims 20-25, wherein the machine learning classifier comprises a random forest, a support vector machine (SVM), a boosting algorithm, a gradient boost method (GBM), an extreme gradient boost method (XGBoost)), and/or a neural network.

27. The method of any of claims 20-26, wherein the method is computer-implemented.

28. A method of detecting disease in a patient, the method comprising the steps of:
obtaining a sample from the patient;
extracting cell-free DNA (cfDNA) from the sample to obtain cfDNA fragments;
performing sequencing on the cfDNA fragments extracted from the sample to generate sequencing reads for the cfDNA fragments;
determining an average nucleotide frequency at start sites and end sites of the cfDNA fragments;
determining a fraction of aberrant fragments in the cfDNA fragments from the sample;
determining a fraction of short fragments in the cfDNA fragments from the sample;
inputting the average nucleotide frequency, the fraction of aberrant fragments, and the fraction of short fragments into a machine learning classifier trained using genomic data from both healthy and diseased subjects; and
determining presence of the disease in the patient based on output of the machine learning classifier.

29. The method of claim 28, wherein the cfDNA fragments having a length of less than 300 bp, less than 275 bp, less than 250 bp, less than 225 bp, less than 200 bp, less than 175 bp, less than 150 bp, less than 125 bp, or less than 100 bp are considered short fragments.

30. The method of claim 28, wherein the cfDNA fragments having a length of less than a selected threshold length are considered short fragments.

31. The method of claim 30, wherein the selected threshold length is about 150 bp.

32. The method of any of claims 28-31, wherein the disease is cancer.

33. The method of any of claims 28-32, wherein the machine learning classifier comprises a random forest, a support vector machine (SVM), a boosting algorithm, a gradient boost method (GBM), an extreme gradient boost method (XGBoost)), and/or a neural network.

34. The method of any of claims 28-33, wherein the method is computer-implemented.

35. A method of detecting disease in a patient, the method comprising the steps of:
obtaining a sample from the patient;
extracting cell-free DNA (cfDNA) from the sample to obtain cfDNA fragments;
performing sequencing on the cfDNA fragments extracted from the sample to generate sequencing reads for the cfDNA fragments;
determining an average nucleotide frequency at start sites and end sites of the cfDNA fragments;
inputting the average nucleotide frequency into a machine learning classifier trained using genomic data from both healthy and diseased subjects; and
determining presence of the disease in the patient based on output of the machine learning classifier.

36. The method of claim 35, further comprising training the machine learning classifier using average nucleotide frequency at start sites and end sites in cfDNA from healthy subjects and using average nucleotide frequency at start sites and end sites in cfDNA from diseased subjects.

37. The method of claim 35 or 36, wherein the disease is cancer.

38. The method of any of claims 35-37, wherein the machine learning classifier comprises a random forest, a support vector machine (SVM), a boosting algorithm, a gradient boost method (GBM), an extreme gradient boost method (XGBoost)), and/or a neural network.

39. The method of any of claims 35-38, wherein the method is computer-implemented.

40. A method of detecting disease in a patient, the method comprising the steps of:
obtaining a sample from the patient;
extracting cell-free DNA (cfDNA) from the sample to obtain cfDNA fragments;
performing sequencing on the cfDNA fragments extracted from the sample to generate sequencing reads for the cfDNA fragments;
determining a fraction of aberrant fragments in the cfDNA fragments from the sample;
inputting the fraction of aberrant fragments into a machine learning classifier trained using genomic data from both healthy and diseased subjects; and
determining presence of the disease in the patient based on output of the machine learning classifier.

41. The method of claim 40, further comprising generating the machine learning classifier by training the machine learning classifier using fractions of aberrant fragments in cfDNA from healthy subjects and using fractions of aberrant fragments in cfDNA from diseased subjects.

42. The method of claim 40 or 41, wherein the disease is a specific cancer subtype.

43. The method of any of claim 40 or 41, wherein the disease is cancer.

44. The method of claim 43, wherein the cancer is selected from the group consisting of melanoma, cholangiocarcinoma, glioblastoma, breast cancer, prostate cancer, colorectal cancer, gastric cancer, lung cancer, and ovarian cancer.

45. The method of any of claims 40-44, wherein the sample is plasma, urine, or cerebrospinal fluid.

46. The method of any of claims 40-45, wherein the patient is human.

47. The method of any of claims 40-45, wherein the patient is a dog or a cat.

48. The method of claim 40-45, wherein the healthy and diseased subjects are non-human.

49. The method of claim 48, wherein the healthy and diseased subjects include dogs or cats.

50. The method of any of claims 40-49 wherein the machine learning classifier comprises a random forest, a support vector machine (SVM), a boosting algorithm, a gradient boost method (GBM), an extreme gradient boost method (XGBoost)), and/or a neural network.

51. The method of claim 50, wherein the machine learning classifier comprises a random forest.

52. The method of claim 50 or 51, wherein the machine learning classifier comprises a gradient boosted tree and/or a neural network.

53. The method of any of claims 40-52, wherein the method is computer-implemented.

54. The method of any of claims 1-53, further comprising selecting specific nucleotide frequencies to feed into the machine learning classifier by determining which nucleotide frequencies are most highly correlated with tumor fraction and fraction of aberrant fragments (FAF).

55. The method of any of claims 1-54, wherein the output of the machine learning classifier comprises a probability that the patient has the disease.

56. The method of any of claims 1-55, wherein sequencing of the cfDNA fragments is performed with whole genome sequencing or hybrid capture sequencing.

57. A non-transitory computer-readable storage device storing computer executable instructions that when executed by a computer control the computer to perform a method for detecting disease in a patient, the method comprising:
determining an average nucleotide frequency at start sites and end sites of cfDNA fragments extracted from a sample from the patient;
determining a fraction of aberrant fragments in the cfDNA fragments from the sample;
inputting the average nucleotide frequency and the fraction of aberrant fragments into a machine learning classifier trained using genomic data from both healthy and diseased subjects; and
determining presence of the disease in the patient based on output of the machine learning classifier.

58. A non-transitory computer-readable storage device storing computer executable instructions that when executed by a computer control the computer to perform a method for detecting disease in a patient, the method comprising:
determining a nucleotide frequency at start sites and end sites of cfDNA fragments extracted from a sample from the patient;
generating a nucleotide frequency vector from the nucleotide frequency at start sites and end sites;
determining a fraction of aberrant fragments in the cfDNA fragments from the sample;
inputting the nucleotide frequency vector and the fraction of aberrant fragments into a random forest classifier trained using genomic data from both healthy and diseased subjects; and
determining presence of the disease in the patient based on output of the random forest classifier.

59. A non-transitory computer-readable storage device storing computer executable instructions that when executed by a computer control the computer to perform a method for detecting disease in a patient, the method comprising:
determining an average nucleotide frequency at start sites and end sites of cfDNA fragments extracted from a sample from the patient;
determining a fraction of aberrant fragments in the cfDNA fragments from the sample;
determining a fraction of short fragments in the cfDNA fragments from the sample;
inputting the average nucleotide frequency, the fraction of aberrant fragments, and the fraction of short fragments into a machine learning classifier trained using genomic data from both healthy and diseased subjects; and
determining presence of the disease in the patient based on output of the machine learning classifier.

60. A non-transitory computer-readable storage device storing computer executable instructions that when executed by a computer control the computer to perform a method for detecting disease in a patient, the method comprising:
determining an average nucleotide frequency at start sites and end sites of cfDNA fragments extracted from a sample from the patient;
inputting the average nucleotide frequency into a machine learning classifier trained using genomic data from both healthy and diseased subjects; and
determining presence of the disease in the patient based on output of the machine learning classifier.

61. A non-transitory computer-readable storage device storing computer executable instructions that when executed by a computer control the computer to perform a method for detecting disease in a patient, the method comprising:
determining a fraction of aberrant fragments in cfDNA fragments extracted from a sample from the patient;
inputting the fraction of aberrant fragments into a machine learning classifier trained using genomic data from both healthy and diseased subjects; and
determining presence of the disease in the patient based on output of the machine learning classifier.

62. A computer-implemented system comprising: a server comprising at least one processor configured to generate a machine learning classifier that classifies cfDNA fragment data into a disease classification for a disease, wherein the machine learning classifier is generated by:
determining an average nucleotide frequency at start sites and end sites of cfDNA fragments;
determining a fraction of aberrant fragments in the cfDNA fragments; and
inputting average nucleotide frequencies and fractions of aberrant fragments into the machine learning classifier to train the classifier using genomic data from both healthy and diseased subjects.

63. The method of any of claim 57 or 62, wherein the disease is cancer.

64. The method of any of claims 57-63, wherein the machine learning classifier comprises a random forest, a support vector machine (SVM), a boosting algorithm, a gradient boost method (GBM), an extreme gradient boost method (XGBoost), and/or a neural network.

65. The method of claim 64, wherein the machine learning classifier comprises a random forest.

66. The method of claim 64 or 65, wherein the machine learning classifier comprises a gradient boosted tree and/or a neural network.
